(19) United States
(12) Patent Application Publication
(10) Pub. No.: US 2021/0363259 A1
(43) Pub. Date: Nov. 25, 2021

(54) PHARMACEUTICAL COMPOSITIONS AND DOSAGE REGIMENS CONTAINING ANTI-ALPHA(V)BETA(6) ANTIBODIES

(71) Applicant: Biogen MA Inc., Cambridge, MA (US)

(72) Inventors: Tia Brie Estey, Swampscott, MA (US); Geetha Govindan, Lancaster, MA (US); Sonal Saluja, Shrewsbury, MA (US); Kapil Gupta, Concord, MA (US); Margaret McGrath, Somerville, MA (US)

(21) Appl. No.: 16/640,422

(22) PCT Filed: Aug. 22, 2018
(86) PCT No.: PCT/US2018/047502

§ 371 Date: Feb. 20, 2020

Related U.S. Application Data
(60) Provisional application No. 62/548,772, filed on Aug. 22, 2017.

(51) Int. Cl. C07K 16/28; A61K 47/18; A61K 47/26; A61K 31/4418; A61K 31/496; A61K 47/12; A61K 47/20

(52) U.S. Cl. CPC C07K 16/2842; A61K 47/183; A61K 47/26; A61K 31/4418; A61K 2039/545; A61K 47/12; A61K 47/20; C07K 2317/565; A61K 31/496

(57) ABSTRACT

Formulations and dosage regimens of an anti-αvβ6 antibody or αvβ6-binding fragment thereof are provided. These formulations find use in the treatment of e.g., fibrosis (e.g., idiopathic pulmonary fibrosis), acute lung injury, and acute kidney injury.

### CROSS-REFERENCE TO RELATED APPLICATIONS

This application claims the benefit of priority of U.S. Provisional Appl. No. 62/548,772, filed Aug. 22, 2017, the content of which is incorporated by reference in its entirety herein.

### FIELD

The present application relates generally to pharmaceutical compositions and dosage regimens for clinical use comprising anti-αvβ6 antibodies and uses thereof.

### BACKGROUND

Integrins are a superfamily of cell surface glycoprotein receptors, which bind extracellular matrix proteins and mediate cell-cell and cell-extracellular matrix interactions (generally referred to as cell adhesion events). These receptors are composed of noncovalently associated alpha (α) and beta (β) chains, which combine to give a variety of heterodimeric proteins with distinct cellular and adhesive specificities. Integrins regulate a variety of cellular processes including cellular adhesion, migration, invasion, differentiation, proliferation, apoptosis and gene expression.

The αvβ6 receptor is one member of a family of integrins that are expressed as cell surface heterodimeric proteins. While the αv subunit can form a heterodimer with a variety of β subunits (β1, β3, β5, β6, and β8), the β6 subunit can only be expressed as a heterodimer with the αv subunit. The αvβ6 integrin is known to be a fibronectin-, vitronectin-, latency associated peptide (LAP)-, and tenascin C-binding cell surface receptor, interacting with the extracellular matrix through the RGD tripeptide binding sites thereon. The expression of αvβ6 is restricted to epithelial cells where it is expressed at relatively low levels in healthy tissue and significantly upregulated during development, injury, and wound healing.

As αvβ6's binding to LAP is important in the conversion of TGF-β to its active state, blocking the binding can result in inhibition of αvβ6-mediated activation of TGF-β and the associated fibrotic pathology.

High affinity antagonist antibodies that bind αvβ6 have been shown to be useful in the treatment of TGF-β-associated disorders.

### SUMMARY

This disclosure relates, in part, to compositions containing an anti-αvβ6 antibody or αvβ6-binding fragment thereof and their use in the treatment of, inter alia, fibrosis, acute lung injury, and acute kidney injury.

In one aspect, the disclosure features a pharmaceutical composition comprising an anti-αvβ6 antibody or αvβ6-binding fragment thereof, and arginine hydrochloride (Arg.HCl). The anti-αvβ6 antibody or αvβ6-binding fragment thereof comprises an immunoglobulin heavy chain variable domain (VH) and an immunoglobulin light chain variable domain (VL). In certain instances, the VH comprises VH complementarity determining regions (VH-CDRs), wherein VH-CDR1 comprises or consists of the amino acid sequence set forth in SEQ ID NO:1 or 11; VH-CDR2 comprises or consists of the amino acid sequence set forth in SEQ ID NO:2; and VH-CDR3 comprises or consists of the amino acid sequence set forth in SEQ ID NO:3; and the VL comprises VL-CDRs, wherein VL-CDR1 comprises or consists of the amino acid sequence set forth in SEQ ID NO:4; VL-CDR2 comprises or consists of the amino acid sequence set forth in SEQ ID NO:5; and VL-CDR3 comprises or consists of the amino acid sequence set forth in SEQ ID NO:6. The composition has a pH of 5.2 to 5.7.

In certain embodiments, the composition comprises the anti-αvβ6 antibody or αvβ6-binding fragment thereof at a concentration of 50 mg/ml to 200 mg/ml. In other embodiments, the composition comprises the anti-αvβ6 antibody or αvβ6-binding fragment thereof at a concentration of 100 mg/ml to 175 mg/ml. In other embodiments, the composition comprises the anti-αvβ6 antibody or αvβ6-binding fragment thereof at a concentration of 125 mg/ml to 175 mg/ml. In yet other embodiments, the composition comprises the anti-αvβ6 antibody or αvβ6-binding fragment thereof at a concentration of 150 mg/ml.

In certain embodiment, the composition comprises Arg.HCl at a concentration of 50 mM to 250 mM. In another embodiment, the composition comprises Arg.HCl at a concentration of 100 mM to 200 mM. In other embodiments, the composition comprises Arg.HCl at a concentration of 125 mM to 175 mM. In yet another embodiment, wherein the composition comprises Arg.HCl at a concentration of 150 mM.

In certain embodiment, the composition comprises methionine. In some instances, the composition comprises methionine at a concentration of 0.5 mM to 30 mM. In other instances, wherein the composition comprises methionine at a concentration of 1 mM to 10 mM. In yet other instances, the composition comprises methionine at a concentration of 5 mM.

In certain embodiment, the composition comprises Polysorbate-80 (PS80). In some instances, the composition comprises PS80 at a concentration of 0.01% to 0.1%. In other instances, the composition comprises PS80 at a concentration of 0.03% to 0.08%. In yet other instances, the composition comprises PS80 at a concentration of 0.05%.

In certain embodiment, the composition comprises sodium citrate and citric acid. In certain instances, the composition comprises sodium citrate and citric acid at a concentration of 5 mM to 30 mM. In other instances, the composition comprises sodium citrate and citric acid at a concentration of 15 mM to 25 mM. In other instances, the composition comprises sodium citrate and citric acid at a concentration of 20 mM.

In certain embodiment, the composition has a pH of 5.3 to 5.6. In one embodiment, the composition has a pH of 5.5.

In certain embodiments, the composition comprises a thiol-containing antioxidant. In some cases, the thiol-containing antioxidant is selected from the group consisting of GSH, GSSG, the combination of GSH and GSSG, cystine, cysteine, and the combination of cysteine and cystine. In one instance, the thiol-containing antioxidant is GSH. In one instance, the thiol-containing antioxidant is GSSG. In one instance, the thiol-containing antioxidant is GSH and GSSG. In one instance, the thiol-containing antioxidant is cysteine. In one instance, the thiol-containing antioxidant is cystine. In one instance, the thiol-containing antioxidant is cysteine and cystine. In certain embodiments, the thiol-containing antioxidant is present in the composition at a concentration of 0.02 mM to 2 mM. In some cases, the thiol-containing antioxidant is present in the composition at a concentration of 0.2 mM. In other cases, the thiol-containing antioxidant is present in the composition at a concentration of 0.4 mM. In yet other cases, the thiol-containing antioxidant is present in the composition at a concentration of 1.0 mM. In cases where the thiol-containing antioxidant is GSH and GSSG, the former is present at a concentration of 0.4 mM and the latter at a concentration of 0.2 mM. In cases where the thiol-containing antioxidant is cysteine and cystine, the former is present at a concentration of 0.4 mM and the latter at a concentration of 0.2 mM.

In some embodiments, the pharmaceutical composition comprises the anti-αvβ6 antibody or the αvβ6-binding fragment thereof at a concentration of 125 mg/ml to 175 mg/ml; Arg.HCl at a concentration of 125 mM to 175 mM; methionine at a concentration of 1 mM to 10 mM; sodium citrate and citric acid at a concentration of 15 mM to 25 mM; and PS80 at a concentration of 0.03% to 0.08%. The composition has a pH of 5.3 to 5.7.

In some embodiments, the pharmaceutical composition comprises the anti-αvβ6 antibody or the αvβ6-binding fragment thereof at a concentration of 125 mg/ml to 175 mg/ml; Arg.HCl at a concentration of 125 mM to 175 mM; methionine at a concentration of 1 mM to 10 mM; sodium citrate and citric acid at a concentration of 15 mM to 25 mM; a thiol-containing antioxidant at a concentration of 0.02 mM to 2 mM; and PS80 at a concentration of 0.03% to 0.08%. The composition has a pH of 5.3 to 5.7.

In some embodiments, the pharmaceutical composition comprises the anti-αvβ6 antibody or the αvβ6-binding fragment thereof at a concentration of 125 mg/ml to 175 mg/ml; Arg.HCl at a concentration of 125 mM to 175 mM; sodium citrate buffer (sodium citrate and citric acid) at a concentration of 15 mM to 25 mM; a thiol-containing antioxidant at a concentration of 0.02 mM to 2 mM; and PS80 at a concentration of 0.03% to 0.08%. The composition has a pH of 5.3 to 5.7.

In some embodiments, the pharmaceutical composition comprises the anti-αvβ6 antibody or the αvβ6-binding fragment thereof at a concentration of 150 mg/ml; Arg.HCl at a concentration of 150 mM; methionine at a concentration of 5 mM; sodium citrate and citric acid at a concentration of 20 mM; and PS80 at a concentration of 0.03% to 0.08%. The composition has a pH of 5.5.

In some embodiments, the pharmaceutical composition comprises the anti-αvβ6 antibody or the αvβ6-binding fragment thereof at a concentration of 150 mg/ml; Arg.HCl at a concentration of 150 mM; methionine at a concentration of 5 mM; sodium citrate and citric acid at a concentration of 20 mM; GSH or cysteine at a concentration of 0.4 mM; and PS80 at a concentration of 0.03% to 0.08%. The composition has a pH of 5.5.

In certain embodiments, the VH consists of a sequence at least 80%, at least 90%, or 100% identical to SEQ ID NO:7 and the VL consists of a sequence at least 80%, at least 90%, or 100% identical to SEQ ID NO:8.

In certain embodiments, the heavy chain consists of a sequence at least 80%, at least 90%, or 100% identical to SEQ ID NO:9 and the light chain consists of a sequence at least 80%, at least 90%, or 100% identical to SEQ ID NO:10;

The disclosure also features methods of treating an αvβ6-mediated condition in a human subject in need thereof. The method comprises administering to the human subject a pharmaceutical composition described herein. In certain instances, the αvβ6-mediated condition is fibrosis. In specific embodiments, the fibrosis is lung fibrosis, kidney fibrosis, liver fibrosis, or cardiac fibrosis. In a particular embodiment, the fibrosis is idiopathic pulmonary fibrosis. In another instance, the αvβ6-mediated condition is acute lung injury. In another instance, the αvβ6-mediated condition is acute kidney injury. In certain embodiments, the pharmaceutical composition is administered subcutaneously to the human subject. In some instances, the anti-αvβ6 antibody or αvβ6-binding fragment thereof of the pharmaceutical composition is administered to the human subject at a dose of 40 mg to 64 mg once weekly. In some instances, the anti-αvβ6 antibody or αvβ6-binding fragment thereof of the pharmaceutical composition is administered to the human subject at a dose of 40 mg once weekly. In some instances, the anti-αvβ6 antibody or αvβ6-binding fragment thereof of the pharmaceutical composition is administered to the human subject at a dose of 48 mg once weekly. In some instances, the anti-αvβ6 antibody or αvβ6-binding fragment thereof of the pharmaceutical composition is administered to the human subject at a dose of 56 mg once weekly. In some instances, the anti-αvβ6 antibody or αvβ6-binding fragment thereof of the pharmaceutical composition is administered to the human subject at a dose of 64 mg once weekly. In some instances, the anti-αvβ6 antibody or αvβ6-binding fragment thereof of the pharmaceutical composition is administered to the human subject at a dose of 0.5 mg/kg to 0.8 mg/kg once weekly. In certain cases, the anti-αvβ6 antibody or αvβ6-binding fragment thereof of the pharmaceutical composition is administered to the human subject at a dose of 0.5 mg/kg once weekly. In certain cases, the anti-αvβ6 antibody or αvβ6-binding fragment thereof of the pharmaceutical composition is administered to the human subject at a dose of 0.6 mg/kg once weekly. In certain cases, the anti-αvβ6 antibody or αvβ6-binding fragment thereof of the pharmaceutical composition is administered to the human subject at a dose of 0.7 mg/kg once weekly. In other cases, the anti-αvβ6 antibody or αvβ6-binding fragment thereof of the pharmaceutical composition is administered to the human subject at a dose of 0.8 mg/kg once weekly.

In another aspect, the disclosure provides a method of treating an αvβ6-mediated condition selected from the group consisting of fibrosis, acute lung injury, and acute kidney injury in a human subject in need thereof. The method comprises administering subcutaneously to the human subject an anti-αvβ6 antibody or αvβ6-binding fragment thereof at a dose of 40 mg to 64 mg once every week. The anti-αvβ6 antibody or αvβ6-binding fragment thereof comprises a VH and a VL. The VH comprises VH-CDRs, wherein VH-CDR1 comprises or consists of the amino acid sequence set forth in SEQ ID NO:1 or 11; VH-CDR2 comprises or consists of the amino acid sequence set forth in SEQ ID NO:2; and VH-CDR3 comprises or consists of the amino acid sequence set forth in SEQ ID NO:3; and

VL-CDRs, wherein VL-CDR1 comprises or consists of the amino acid sequence set forth in SEQ ID NO:4; VL-CDR2 comprises or consists of the amino acid sequence set forth in SEQ ID NO:5; and VL-CDR3 comprises or consists of the amino acid sequence set forth in SEQ ID NO:6. In certain instances, the dose is 40 mg once every week. In certain instances, the dose is 48 mg once every week. In certain instances, the dose is 56 mg once every week. In certain instances, the dose is 64 mg once every week. In certain instances, the human subject is administered at least 4 doses of the anti-αvβ6 antibody or antigen-binding fragment thereof. In other instances, the human subject is administered at least 7 doses of the anti-αvβ6 antibody or antigen-binding fragment thereof. In yet other instances, the human subject is administered at least 10 doses of the anti-αvβ6 antibody or antigen-binding fragment thereof. In some cases, the VH consists of a sequence at least 80%, at least 90%, or 100% identical to SEQ ID NO:7 and the VL consists of a sequence at least 80%, at least 90%, or 100% identical to SEQ ID NO:8. In some instances, the anti-αvβ6 antibody comprises an immunoglobulin heavy chain and an immunoglobulin light chain, wherein the heavy chain consists of a sequence at least 80%, at least 90%, or 100% identical to SEQ ID NO:9 and the light chain consists of a sequence at least 80%, at least 90%, or 100% identical to SEQ ID NO:10. In certain instances, the condition is fibrosis. In specific embodiments, the fibrosis is lung fibrosis, kidney fibrosis, liver fibrosis, or cardiac fibrosis. In a particular embodiment, the fibrosis is idiopathic pulmonary fibrosis. In another instance, the condition is acute lung injury. In another instance, the condition is acute kidney injury.

In another aspect, the disclosure features a syringe or pump comprising a sterile preparation of a pharmaceutical composition described herein, wherein the syringe or pump is adapted for subcutaneous administration of the anti-αvβ6 antibody or αvβ6-binding fragment thereof at a fixed dose of 40 mg, 48 mg, 56 mg, or 64 mg. In certain instances, the syringe or pump comprises 0.5 to 5.0 mL of a sterile preparation of a pharmaceutical composition described herein. In certain instances, the syringe or pump comprises 0.5 to 1.0 mL of a sterile preparation of a pharmaceutical composition described herein. In a specific embodiment, the disclosure features a syringe or pump comprising 0.8 ml of a 70 mg/ml formulation comprising the anti-αvβ6 antibody or αvβ6-binding fragment thereof. In a specific embodiment, the disclosure features a syringe or pump comprising 0.8 ml of formulation comprising the anti-αvβ6 antibody or αvβ6-binding fragment thereof at a fixed dose of 56 mg. In certain instances, the pump is an LVSC pump.

In another aspect, the disclosure features a syringe or pump comprising a sterile preparation of an anti-αvβ6 antibody or αvβ6-binding fragment thereof. The syringe or pump is adapted for subcutaneous administration of the anti-αvβ6 antibody or αvβ6-binding fragment thereof at a fixed dose of 40 mg, 48 mg, 56 mg, or 64 mg. The anti-αvβ6 antibody or αvβ6-binding fragment thereof comprises a VH and a VL. The VH-CDRs comprise VH-CDR1 consisting of the amino acid sequence set forth in SEQ ID NO:1 or 11; VH-CDR2 consisting of the amino acid sequence set forth in SEQ ID NO:2; and VH-CDR3 consisting of the amino acid sequence set forth in SEQ ID NO:3. The VL-CDRs comprise VL-CDR1 consisting of the amino acid sequence set forth in SEQ ID NO:4; VL-CDR2 consisting of the amino acid sequence set forth in SEQ ID NO:5; and VL-CDR3 consisting of the amino acid sequence set forth in SEQ ID NO:6. In certain instances, the VH consists of a sequence at least 80%, at least 90%, or 100% identical to SEQ ID NO:7 and the VL consists of a sequence at least 80%, at least 90%, or 100% identical to SEQ ID NO:8. In some instances, the anti-αvβ6 antibody comprises an immunoglobulin heavy chain and an immunoglobulin light chain, wherein the heavy chain consists of a sequence at least 80%, at least 90%, or 100% identical to SEQ ID NO:9 and the light chain consists of a sequence at least 80%, at least 90%, or 100% identical to SEQ ID NO:10.

In another aspect, the disclosure features a combination treatment regimen comprising a pharmaceutical composition described herein and prifenidone.

In yet another aspect, the disclosure features a combination treatment regimen comprising a pharmaceutical composition described herein and nintedanib.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF THE DRAWINGS

FIG. 1A is a graph depicting the % total aggregation as determined by size exclusion chromatography (SEC) of 150 g/L STX-100 formulations with different excipients.

FIG. 1B is a bar graph showing the total sub-visible particles per mL of 150 g/L STX-100 formulations with different excipients. For each formulation tested, T=0 is depicted as the left bar and T=4 wk is depicted as the right bar.

FIG. 1C is a bar graph showing the viscosity at ambient temperature of 150 g/L STX-100 formulations with different excipients.

FIG. 2 is a bar graph showing the results of a pH-arginine screening study. For each formulation tested, T=0 is depicted as the left bar and T=1M 40° C. is depicted as the right bar.

FIG. 3 is a graph showing the effect of GSH on the aggregation of STX-100 formulations at 25° C. (top) and 40° C. (bottom). The STX-100 formulations contain 150 mg/ml of STX-100, 20 mM citrate/citric acid, 150 mM arginine hydrochloride, 5 mM methionine, 0.05% PS80 and a pH of 5.5, and either no GSH or 0.4 mM GSH.

FIG. 4 provides graphs depicting the percentage of HMW species of SB4 (BENEPALI®, an etanercept biosimilar referencing Enbrel®) formulation (50 mg/ml SB4; 10 mM sodium phosphate; 140 mM NaCl; 1% sucrose, pH 6.2) with or without GSH (0.4 mM) at 25° C. and 40° C.

FIG. 5 provides graphs depicting the percentage of HMW species of an anti-αvβ5 integrin antibody (STX200) formulation (50 mg/ml antibody; 20 mM histidine; 5% sorbitol; 0.05% PS80, pH 6.5) with or without GSH (0.4 mM) at 25° C. and 40° C.

### DETAILED DESCRIPTION

This application provides pharmaceutical compositions and dosage regimens of anti-αvβ6 antibodies and αvβ6-binding fragments thereof and their use in the treatment of diseases such as, but not limited to, fibrosis, acute lung injury, acute kidney injury, and cancer.

αvβ6

αvβ6 is an integrin that is expressed on epithelial cells. It can bind to several ligands including fibronectin, vitronectin, cytotactin, tenascin, and the latency associated peptide-1 and -3 (LAP1 and LAP3)—the N-terminal 278 amino acids of the latent precursor form of TGF-β1—through a direct interaction with an arginine-glycine-aspartate (“RGD”) motif. The TGF-β cytokine is synthesized as a latent complex which has the N-terminal LAP noncovalently associated with the mature active C-terminal TGF-β cytokine. The latent TGF-β complex cannot bind to its cognate receptor and thus is not biologically active until converted to an active form. αvβ6 binding to LAP1 or LAP3 leads to activation of the latent precursor form of TGF-β1 and TGF-β3 as a result of a conformational change in the latent complex allowing TGF-β to bind to its receptor. Thus, upregulated expression of αvβ6 can lead to local activation of TGF-β, which in turn can activate a cascade of events downstream events. The TGF-β1 cytokine is a pleiotropic growth factor that regulates cell proliferation, differentiation, and immune responses.

The amino acid sequence of human integrin αv (UniProtKB—P06756 (ITAV HUMAN) is shown below (the 30 aa signal peptide sequence is underlined):

```
(SEQ ID NO: 12)
                10 20 30 40
MAFPPRRRLR LGPRGLPLLL SGLLLPLCRA FNLDVDSPAE
                50 60 70 80
YSGPEGSYFG FAVDFFVPSA SSRMFLLVGA PKANTTQPGI
                90 100 110 120
VEGGQVLKCD WSSTRRCQPI EFDATGNRDY AKDDPLEFKS
              130 140 150 160
HQWFGASVRS KQDKILACAP LYHWRTEMKQ EREPVGTCFL
              170 180 190 200
QDGTKTVEYA PCRSQDIDAD GQGFCQGGFS IDFTKADRVL
              210 220 230 240
LGGPGSFYWQ GQLISDQVAE IVSKYDPNVY SIKYNNQLAT
              250 260 270 280
RTAQAIFDDS YLGYSVAVGD FNGDGIDDFV SGVPRAARTL
              290 300 310 320
GMVYIYDGKN MSSLYNFTGE QMAAYFGFSV AATDINGDDY
              330 340 350 360
ADVFIGAPLF MDRGSDGKLQ EVGQVSVSLQ RASGDFQTTK
              370 380 390 400
LNGFEVFARF GSAIAPLGDL DQDGFNDIAI AAPYGGEDKK
              410 420 430 440
GIVYIFNGRS TGLNAVPSQI LEGQWAARSM PPSFGYSMKG
              450 460 470 480
ATDIDKNGYP DLIVGAFGVD RAILYRARPV ITVNAGLEVY
              490 500 510 520
PSILNQDNKT CSLPGTALKV SCFNVRFCLK ADGKGVLPRK
              530 540 550 560
LNFQVELLLD KLKQKGAIRR ALFLYSRSPS HSKNMTISRG
              570 580 590 600
GLMQCEELIA YLRDESEFRD KLTPITIFME YRLDYRTAAD
              610 620 630 640
TTGLQPILNQ FTPANISRQA HILLDCGEDN VCKPKLEVSV
              650 660 670 680
DSDQKKIYIG DDNPLTLIVK AQNQGEGAYE AELIVSIPLQ
              690 700 710 720
ADFIGVVRNN EALARLSCAF KTENQTRQVV CDLGNPMKAG
              730 740 750 760
TQLLAGLRFS VHQQSEMDTS VKFDLQIQSS NLFDKVSPVV
              770 780 790 800
SHKVDLAVLA AVEIRGVSSP DHVFLPIPNW EHKENPETEE
              810 820 830 840
DVGPVVQHIY ELRNNGPSSF SKAMLHLQWP YKYNNNTLLY
              850 860 870 880
ILHYDIDGPM NCTSDMEINP LRIKISSLQT TEKNDTVAGQ
              890 900 910 920
GERDHLITKR DLALSEGDIH TLGCGVAQCL KIVCQVGRLD
              930 940 950 960
RGKSAILYVK SLLWTETFMN KENQNHSYSL KSSASFNVIE
              970 980 990 1000
FPYKNLPIED ITNSTLVTTN VTWGIQPAPM PVPVWVIILA
            1010 1020 1030 1040
VLAGLLLLAV LVFVMYRMGF FKRVRPPQEE QEREQLQPHE
NGEGNSET
```

The mature αv protein corresponds to amino acids 31-1048 of SEQ ID NO:12.

The amino acid sequence of human integrin β6 (UniProtKB—P18564 (ITB6 HUMAN) is provided below (the 21 aa signal peptide sequence is underlined):

```
(SEQ ID NO: 13)
                10 20 30 40
MGIELLCLFF LFLGRNDHVQ GGCALGGAET CEDCLLIGPQ
                50 60 70 80
CAWCAQENFT HPSGVGERCD TPANLLAKGC QLNFIENPVS
                90 100 110 120
QVEILKNKPL SVGRQKNSSD IVQIAPQSLI LKLRPGGAQT
              130 140 150 160
LQVHVRQTED YPVDLYYLMD LSASMDDDLN TIKELGSRLS
              170 180 190 200
KEMSKLTSNF RLGFGSFVEK PVSPFVKTTP EEIANPCSSI
              210 220 230 240
PYFCLPTFGF KHILPLTNDA ERFNEIVKNQ KISANIDTPE
              250 260 270 280
GGFDAIMQAA VCKEKIGWRN DSLHLLVFVS DADSHFGMDS
              290 300 310 320
KLAGIVIPND GLCHLDSKNE YSMSTVLEYP TIGQLIDKLV
              330 340 350 360
QNNVLLIFAV TQEQVHLYEN YAKLIPGATV GLLQKDSGNI
              370 380 390 400
LQLIISAYEE LRSEVELEVL GDTEGLNLSF TAICNNGTLF
              410 420 430 440
QHQKKCSHMK VGDTASFSVT VNIPHCERRS RHIIIKPVGL
              450 460 470 480
GDALELLVSP ECNCDCQKEV EVNSSKCHHG NGSFQCGVCA
              490 500 510 520
CHPGHMGPRC ECGEDMLSTD SCKEAPDHPS CSGRGDCYCG
              530 540 550 560
QCICHLSPYG NIYGPYCQCD NFSCVRHKGL LCGGNGDCDC
              570 580 590 600
GECVCRSGWT GEYCNCTTST DSCVSEDGVL CSGRGDCVCG
              610 620 630 640
KCVCTNPGAS GPTCERCPTC GDPCNSKRSC IECHLSAAGQ
              650 660 670 680
AREECVDKCK LAGATISEEE DFSKDGSVSC SLQGENECLI
              690 700 710 720
TFLITTDNEG KTIIHSINEK DCPKPPNIPM IMLGVSLAIL
              730 740 750 760
LIGVVLLCIW KLLVSFHDRK EVAKFEAERS KAKWQTGTNP
              770 780
LYRGSTSTFK NVTYKHREKQ KVDLSTDC
```

The mature β6 protein corresponds to amino acids 22-788 of SEQ ID NO:13.

The antibodies described herein can bind specifically to the αvβ6 protein having the amino acid sequence set forth in positions 31-1048 of SEQ ID NO:12 and the amino acid sequence set forth in positions 22-788 of SEQ ID NO:13. In some embodiments, the antibodies described herein can bind specifically to the β6 protein having the amino acid sequence set forth in positions 22-788 of SEQ ID NO:13.

### Anti-αvβ6 Antibodies

In some embodiments, the anti-αvβ6 antibody or αvβ6-binding fragment thereof used in the compositions and methods described herein comprises the three heavy chain variable domain complementarity determining regions (CDRs) of an antibody referred to as “STX-100”. In some embodiments, the anti-αvβ6 antibody or αvβ6-binding fragment thereof comprises the three light chain variable domain CDRs of STX-100. In still other embodiments, the anti-αvβ6 antibody or αvβ6-binding fragment thereof comprises the three heavy chain variable domain CDRs and the three light chain variable domain CDRs of STX-100. The CDRs can be based on any CDR definition known in the art, e.g., the definitions of Kabat, Chothia, Chothia from Abysis, enhanced Chothia/AbM, or based on the contact definition. Exemplary CDR sequences of STX-100 (according to Kabat) are provided in Table 1 below.

TABLE 1
Sequences of the Kabat CDRs of STX-100

| Domain | CDR |
|---|---|
| VH CDR1 | RYVMS (SEQ ID NO: 1) |
| VH CDR2 | SISSGGRMYYPDTVKG (SEQ ID NO: 2) |
| VH CDR3 | GSIYDGYYVFPY (SEQ ID NO: 3) |
| VL CDR1 | SASSSVSSSYLY (SEQ ID NO: 4) |
| VL CDR2 | STSNLAS (SEQ ID NO: 5) |
| VL CDR3 | HQWSTYPPT (SEQ ID NO: 6) |

In some aspects, the anti-αvβ6 antibody or αvβ6-binding fragment thereof comprises of a VH CDR1 comprising or consisting of the amino acid sequence set forth in SEQ ID NO:1 or GFTFSRYVMS (SEQ ID NO:11), a VH CDR2 comprising or consisting of the amino acid sequence set forth in SEQ ID NO:2; and a VH CDR3 comprising or consisting of the amino acid sequence set forth in SEQ ID NO:3. In some embodiments, the anti-αvβ6 antibody or αvβ6-binding fragment thereof comprises a VL CDR1 comprising or consisting of the amino acid sequence set forth in SEQ ID NO:4, a VL CDR2 comprising or consisting of the amino acid sequence set forth in SEQ ID NO:5; and a VL CDR3 comprising or consisting of the amino acid sequence set forth in SEQ ID NO:6.

In certain aspects, the anti-αvβ6 antibody or αvβ6-binding fragment thereof comprises the CDRs comprising the amino acid sequences set forth in SEQ ID NOs:1 to 6. In certain aspects, the anti-αvβ6 antibody or αvβ6-binding fragment thereof comprises the CDRs comprising the amino acid sequences set forth in SEQ ID NOs:11, 2, 3, 4, 5, and 6. In certain aspects, the anti-αvβ6 antibody or αvβ6-binding fragment thereof comprises the CDRs consisting of the amino acid sequences set forth in SEQ ID NOs:1 to 6. In certain aspects, the anti-αvβ6 antibody or αvβ6-binding fragment thereof comprises the CDRs consisting of the amino acid sequences set forth in SEQ ID NOs:11, 2, 3, 4, 5, and 6.

STX-100 is a humanized humanIgG1/human kappa monoclonal antibody that specifically binds to the integrin αvβ6.

The heavy chain variable domain (VH) of STX-100 comprises or consists of the following amino acid sequence (VH CDRs (Kabat definition) bolded):

| (SEQ ID NO: 7) | |
|---|---|
| 1 | EVQLVESGGG LVQPGGSLRL SCAASGFTFS RYVMSWVRQA PGKGLEWVAS |
| 51 | ISSGGRMYYP DTVKGRFTIS RDNAKNSLYL QMNSLRAEDT AVYYCARGSI |
| 101 | YDGYYVFPYW GQGTLVTVSS |

The light chain variable domain (VL) of STX-100 comprises or consists of the following amino acid sequence (VL CDRs (Kabat definition) bolded):

| (SEQ ID NO: 8) | |
|---|---|
| 1 | EIVLTQSPAT LSLSPGERAT LSCSASSSVS SSYLYWYQQK PGQAPRLLIY |
| 51 | STSNLASGIP ARFSGSGSGT DFTLTISSLE PEDFAVYYCH QWSTYPPTFG |
| 101 | GGTKVEIK |

In certain embodiments, the anti-αvβ6 antibody or αvβ6-binding fragment thereof comprises a VH comprising or consisting of the amino acid sequence set forth in SEQ ID NO:7. In some embodiments, the anti-αvβ6 antibody or αvβ6-binding fragment thereof selectively binds to αvβ6 and comprises a VH domain that is at least 70%, 75%, 80%, 85%, 90%, 91%, 92%, 93%, 94%, 95%, 96%, 97%, 98%, 99% or more identical to the amino acid sequence of the VH domain of STX-100 (SEQ ID NO:7), or differs at least at 1 to 5 amino acid residues, but at fewer than 40, 30, 20, 15, or 10, residues, from SEQ ID NO:7. In some embodiments, these anti-αvβ6 antibody or αvβ6-binding fragments thereof blocks the binding of αvβ6 to its ligand, latency associated peptide (LAP), as determined by blocking of ligand binding either to purified hsαvβ6 or to β6-expressing cells. In some embodiments, these anti-αvβ6 antibody or αvβ6-binding fragments thereof have one or more (e.g., one, two, three, four) of these properties: (i) specifically bind with high affinity to αvβ6; (ii) inhibit the binding of αvβ6 to LAP, fibronectin, vitronectin, or tenascin with an IC50 value lower than that of the 10D5 antibody (WO 99/07405); (iii) block or inhibit activation of TGF-0; (iv) specifically bind to the β6 subunit; and (v) recognize αvβ6 in immunostaining procedures such as immunostaining of paraffin-embedded tissues.

In certain embodiments, the anti-αvβ6 antibody or αvβ6-binding fragment thereof comprises a VL comprising or consisting of the amino acid sequence set forth in SEQ ID NO:8. In some embodiments, the anti-αvβ6 antibody or αvβ6-binding fragment thereof selectively binds to αvβ6 and comprises a VL domain that is at least 70%, 75%, 80%, 85%, 90%, 91%, 92%, 93%, 94%, 95%, 96%, 97%, 98%, 99% or more identical to the amino acid sequence of the VL domain of STX-100 (SEQ ID NO:8), or differs at least at 1 to 5 amino acid residues, but at fewer than 40, 30, 20, 15, or 10, residues, from SEQ ID NO:8. In some embodiments, these anti-αvβ6 antibody or αvβ6-binding fragments thereof blocks the binding of αvβ6 to its ligand, latency associated peptide (LAP), as determined by blocking of ligand binding either to purified hsαvβ6 or to β6-expressing cells. In some embodiments, these anti-αvβ6 antibody or αvβ6-binding fragments thereof have one or more (e.g., one, two, three, four) of these properties: (i) specifically bind with high affinity to αvβ6; (ii) inhibit the binding of αvβ6 to LAP, fibronectin, vitronectin, or tenascin with an IC50 value lower than that of the 10D5 antibody (WO 99/07405); (iii) block or inhibit activation of TGF-β; (iv) specifically bind to the β6 subunit; and (v) recognize αvβ6 in immunostaining procedures such as immunostaining of paraffin-embedded tissues.

In some embodiments, the anti-αvβ6 antibody or αvβ6-binding fragment thereof comprises a VH having the amino acid sequence set forth in SEQ ID NO:7 and a VL having the amino acid sequence set forth in SEQ ID NO:8. In some embodiments, the anti-αvβ6 antibody or αvβ6-binding fragment thereof selectively binds to αvβ6 and comprises (i) a VH domain that is at least 70%, 75%, 80%, 85%, 90%, 91%, 92%, 93%, 94%, 95%, 96%, 97%, 98%, 99% or more identical to the amino acid sequence of the VH domain of STX-100 (SEQ ID NO:7), and (ii) a VL domain that is at least 70%, 75%, 80%, 85%, 90%, 91%, 92%, 93%, 94%, 95%, 96%, 97%, 98%, 99% or more identical to the amino acid sequence of the VL domain of STX-100 (SEQ ID NO:8); or differs at least at 1 to 5 amino acid residues, but at fewer than 40, 30, 20, 15, or 10, residues, from SEQ ID NO:7 and/or SEQ ID NO:8. In some embodiments, these anti-αvβ6 antibody or αvβ6-binding fragments thereof blocks the binding of αvβ6 to its ligand, latency associated peptide (LAP), as determined by blocking of ligand binding either to purified hsαvβ6 or to β6-expressing cells. In some embodiments, these anti-αvβ6 antibody or αvβ6-binding fragments thereof have one or more (e.g., one, two, three, four) of these properties: (i) specifically bind with high affinity to αvβ6; (ii) inhibit the binding of αvβ6 to LAP, fibronectin, vitronectin, or tenascin with an IC50 value lower than that of the 10D5 antibody (WO 99/07405); (iii) block or inhibit activation of TGF-β; (iv) specifically bind to the β6 subunit; and (v) recognize αvβ6 in immunostaining procedures such as immunostaining of paraffin-embedded tissues.

An antibody consisting of the mature heavy chain (SEQ ID NO:9) and the mature light chain (SEQ ID NO:10) listed below is termed “STX-100” or “BG00011” or “BG11”. STX-100 is an IgG1/kappa antibody.

Mature STX-100 Heavy Chain (HC) [H-CDR1, H-CDR2, and H-CDR3 are bolded; constant region underlined; N-linked glycosylation site bolded & underlined]

| (SEQ ID NO: 9) | |
|---|---|
| 1 | EVQLVESGGG LVQPGGSLRL SCAASGFTFS RYVMSWVRQA PGKGLEWVAS |
| 51 | ISSGGRMYYP DTVKGRFTIS RDNAKNSLYL QMNSLRAEDT AVYYCARGSI |
| 101 | YDGYYVFPYW GQGTLVTVSS ASTKGPSVFP LAPSSKSTSG GTAALGCLVK |
| 151 | DYFPEPVTVS WNSGALTSGV HTFPAVLQSS GLYSLSSVVT VPSSSLGTQT |
| 201 | YICNVNHKPS NTKVDKKVEP KSCDKTHTCP PCPAPELLGG PSVFLFPPKP |
| 251 | KDTLMISRTP EVTCVVVDVS HEDPEVKFNW YVDGVEVHNA KTKPREEQYN |
| 301 | STYRVVSVLT VLHQDWLNGK EYKCKVSNKA LPAPIEKTIS KAKGQPREPQ |
| 351 | VYTLPPSRDE LTKNQVSLTC LVKGFYPSDI AVEWESNGQP ENNYKTTPPV |
| 401 | LDSDGSFFLY SKLTVDKSRW QQGNVFSCSV MHEALHNHYT QKSLSLSPG |

Mature STX-100 Light Chain (LC) [L-CDR1, L-CDR2, and L-CDR3 are bolded; constant region underlined]

| (SEQ ID NO: 10) | |
|---|---|
| 1 | EIVLTQSPAT LSLSPGERAT LSCSASSSVS SSYLYWYQQK PGQAPRLLIY |
| 51 | STSNLASGIP ARFSGSGSGT DFTLTISSLE PEDFAVYYCH QWSTYPPTFG |
| 101 | GGTKVEIKRT VAAPSVFIFP PSDEQLKSGT ASVVCLLNNF YPREAKVQWK |
| 151 | VDNALQSGNS QESVTEQDSK DSTYSLSSTL TLSKADYEKH KVYACEVTHQ |
| 201 | GLSSPVTKSF NRGEC |

In certain embodiments, the anti-αvβ6 antibody or αvβ6-binding fragment thereof comprises a HC having the amino acid sequence set forth in SEQ ID NO:9. In some embodiments, the anti-αvβ6 antibody or αvβ6-binding fragment thereof selectively binds to αvβ6 and comprises a HC that is at least 70%, 75%, 80%, 85%, 90%, 91%, 92%, 93%, 94%, 95%, 96%, 97%, 98%, 99% or more identical to the amino acid sequence of SEQ ID NO:9, or differs at least at 1 to 5 amino acid residues, but at fewer than 40, 30, 20, 15, or 10, residues, from SEQ ID NO:9. In certain embodiments, the anti-αvβ6 antibody or αvβ6-binding fragment thereof comprises a heavy chain set forth in SEQ ID NO:9, except for 1 to 5 amino acid substitutions in the heavy chain constant region. In some embodiments, these anti-αvβ6 antibodies or αvβ6-binding fragments thereof block the binding of αvβ6 to its ligand, latency associated peptide (LAP), as determined by blocking of ligand binding either to purified hsαvβ6 or to β6-expressing cells. In some embodiments, these anti-αvβ6 antibody or αvβ6-binding fragments thereof have one or more (e.g., one, two, three, four) of these properties: (i) specifically bind with high affinity to αvβ6; (ii) inhibit the binding of αvβ6 to LAP, fibronectin, vitronectin, or tenascin with an IC50 value lower than that of the 10D5 antibody (WO 99/07405); (iii) block or inhibit activation of TGF-β; (iv) specifically bind to the β6 subunit; and (v) recognize αvβ6 in immunostaining procedures such as immunostaining of paraffin-embedded tissues.

In certain embodiments, the anti-αvβ6 antibody or αvβ6-binding fragment thereof comprises a LC having the amino acid sequence set forth in SEQ ID NO:10. In some embodiments, the anti-αvβ6 antibody or αvβ6-binding fragment thereof selectively binds to αvβ6 and comprises a LC that is at least 70%, 75%, 80%, 85%, 90%, 91%, 92%, 93%, 94%, 95%, 96%, 97%, 98%, 99% or more identical to the amino acid sequence of SEQ ID NO:10, or differs at least at 1 to 5 amino acid residues, but at fewer than 40, 30, 20, 15, or 10, residues, from SEQ ID NO:10. In certain embodiments, the anti-αvβ6 antibody or αvβ6-binding fragment thereof comprises a light chain set forth in SEQ ID NO:10, except for 1 to 5 amino acid substitutions in the light chain constant region. In some embodiments, these anti-αvβ6 antibodies or αvβ6-binding fragments thereof block the binding of αvβ6 to its ligand, latency associated peptide (LAP), as determined by blocking of ligand binding either to purified hsαvβ6 or to β6-expressing cells. In some embodiments, these anti-αvβ6 antibody or αvβ6-binding fragments thereof have one or more (e.g., one, two, three, four) of these properties: (i) specifically bind with high affinity to αvβ6; (ii) inhibit the binding of αvβ6 to LAP, fibronectin, vitronectin, or tenascin with an IC50 value lower than that of the 10D5 antibody (WO 99/07405); (iii) block or inhibit activation of TGF-β; (iv) specifically bind to the β6 subunit; and (v) recognize αvβ6 in immunostaining procedures such as immunostaining of paraffin-embedded tissues.

In certain embodiments, the anti-αvβ6 antibody or αvβ6-binding fragment thereof comprises a HC having the amino acid sequence set forth in SEQ ID NO:9 and a LC having the amino acid sequence set forth in SEQ ID NO:10. In some embodiments, the anti-αvβ6 antibody or αvβ6-binding fragment thereof selectively binds to human αvβ6 and comprises (i) a HC that is at least 70%, 75%, 80%, 85%, 90%, 91%, 92%, 93%, 94%, 95%, 96%, 97%, 98%, 99% or more identical to the amino acid sequence of SEQ ID NO:9, or differs at least at 1 to 5 amino acid residues, but at fewer than 40, 30, 20, 15, or 10, residues, from SEQ ID NO:9; and (ii) a LC that is at least 70%, 75%, 80%, 85%, 90%, 91%, 92%, 93%, 94%, 95%, 96%, 97%, 98%, 99% or more identical to the amino acid sequence of SEQ ID NO:10, or differs at least at 1 to 5 amino acid residues, but at fewer than 40, 30, 20, 15, or 10, residues, from SEQ ID NO:10. In some embodiments, these anti-αvβ6 antibodies or αvβ6-binding fragments thereof block the binding of αvβ6 to its ligand, latency associated peptide (LAP), as determined by blocking of ligand binding either to purified hsαvβ6 or to β6-expressing cells. In some embodiments, these anti-αvβ6 antibody or αvβ6-binding fragments thereof have one or more (e.g., one, two, three, four) of these properties: (i) specifically bind with high affinity to αvβ6; (ii) inhibit the binding of αvβ6 to LAP, fibronectin, vitronectin, or tenascin with an IC50 value lower than that of the 10D5 antibody (WO 99/07405); (iii) block or inhibit activation of TGF-β; (iv) specifically bind to the β6 subunit; and (v) recognize αvβ6 in immunostaining procedures such as immunostaining of paraffin-embedded tissues.

In certain embodiments, the anti-αvβ6 antibody is an IgG antibody. In specific embodiments, the anti-αvβ6 antibody has heavy chain constant region chosen from, e.g., IgG1, IgG2, IgG3, IgG4, IgM, IgA1, IgA2, IgD, and IgE. In one embodiment, the anti-αvβ6 antibody is of the human IgG1 isotype. In another embodiment, the anti-αvβ6 antibody is of the human IgG2 isotype. In yet another embodiment, the anti-αvβ6 antibody is of the human IgG3 isotype. In yet another embodiment, the anti-αvβ6 antibody is of the human IgG4 isotype. In further embodiments, the antibody has a light chain constant region chosen from, e.g., a human kappa or human lambda light chain. In a certain embodiment, the anti-αvβ6 antibody is a human IgG1/human kappa antibody. In some cases, the heavy chain constant region is human or a modified form of a human constant region. In certain instances, the human constant region may include at least 1 and up to 2, 3, 4, 5, 6, 7, 8, 9, 10, 11, 12, 13, 14, 15, 16, 17, 18, 19, or 20 substitutions. In a particular embodiment, the modified human Fc region is a modified human IgG1 Fc region. In some cases, the constant region of an anti-αvβ6 antibody is modified by mutation of one or more amino acid residues to impart a desired functional property (e.g., altered effector function or half-life, reduced glycosylation). For example, the N-linked glycosylation site may be substituted to prevent or reduce N-linked glycosylation of Fc region (e.g., human IgG1 Fc region).

In some embodiments, the anti-αvβ6 antibody is a full-length (whole) antibody or substantially full-length. The protein can include at least one, and preferably two, complete heavy chains, and at least one, and preferably two, complete light chains. In some embodiments, the anti-αvβ6 antibody is an αvβ6-binding fragment. In some instances, the αvβ6-binding fragment is a Fab, a Fab′, an F(ab′)2, a Facb, an Fv, a single chain Fv (scFv), a sc(Fv)2, or a diabody.

Antibodies, such as STX-100, or αvβ6-binding fragments thereof can be made, for example, by preparing and expressing synthetic genes that encode the recited amino acid sequences or by mutating human germline genes to provide a gene that encodes the recited amino acid sequences. Moreover, this antibody and other anti-αvβ6 antibodies can be produced, e.g., using one or more of the following methods.

Methods of Producing Antibodies Anti-αvβ6 antibodies or αvβ6-binding fragments can be produced in bacterial or eukaryotic cells. Some antibodies, e.g., Fab's, can be produced in bacterial cells, e.g., E. coli cells. Antibodies can also be produced in eukaryotic cells such as transformed cell lines (e.g., CHO, 293E, COS). In addition, antibodies (e.g., scFv's) can be expressed in a yeast cell such as Pichia (see, e.g., Powers et al., J Immunol Methods. 251:123-35 (2001)), Hanseula, or Saccharomyces. To produce the antibody of interest, a polynucleotide encoding the antibody is constructed, introduced into an expression vector, and then expressed in suitable host cells. Polynucleotides encoding an anti-αvβ6 antibody comprising the VH and/or VL, HC and/or LC of the αvβ6 antibodies described herein would be readily envisioned by the ordinarily skilled artisan. Standard molecular biology techniques are used to prepare the recombinant expression vector, transfect the host cells, select for transformants, culture the host cells and recover the antibody.

If the anti-αvβ6 antibodies or αvβ6-binding fragments is to be expressed in bacterial cells (e.g., E. coli), the expression vector should have characteristics that permit amplification of the vector in the bacterial cells. Additionally, when E. coli such as JM109, DHSα, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NSO myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of an anti-αvβ6 antibody (e.g., STX-100) is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Anti-αvβ6 Antibody Compositions

This disclosure also provides compositions (e.g., pharmaceutical compositions) comprising the anti-αvβ6 antibodies or αvβ6-binding fragments thereof described herein. For example, the anti-αvβ6 antibody compositions comprise an anti-αvβ6 antibody or αvβ6-binding fragment thereof comprising an immunoglobulin heavy chain variable domain (VH) and an immunoglobulin light chain variable domain (VL), wherein the VH comprises the H-CDRs and the VL comprises the L-CDRs of STX-100. In certain instances, the heavy chain CDRs (H-CDRs) comprise or consist of the amino acid sequences set forth in SEQ ID NO:1, SEQ ID NO:2, and SEQ ID NO:3; and the light chain CDRs (L-CDRs) comprise or consist of the amino acid sequences set forth in SEQ ID NO:4, SEQ ID NO:5, and SEQ ID NO:6. In certain instances, the heavy chain CDRs (H-CDRs) comprise or consist of the amino acid sequences set forth in SEQ ID NO:11, SEQ ID NO:2, and SEQ ID NO:3; and the light chain CDRs (L-CDRs) comprise or consist of the amino acid sequences set forth in SEQ ID NO:4, SEQ ID NO:5, and SEQ ID NO:6. In some embodiments, the anti-αvβ6 antibody compositions comprises an anti-αvβ6 antibody or αvβ6-binding fragment thereof comprising (i) a VH comprising or consisting of an amino acid sequence that is at least 85%, 90%, 91%, 92%, 93%, 94%, 95%, 96%, 97%, 98%, 99%, or 100% identical to the amino acid sequence set forth in SEQ ID NO:7; and (ii) a VL comprising or consisting of an amino acid sequence that is at least 85%, 90%, 91%, 92%, 93%, 94%, 95%, 96%, 97%, 98%, 99%, or 100% identical to the amino acid sequence set forth in SEQ ID NO:8. In certain embodiments, the anti-αvβ6 antibody compositions comprises an anti-αvβ6 antibody comprising (i) a heavy chain comprising or consisting of an amino acid sequence that is at least 85%, 90%, 91%, 92%, 93%, 94%, 95%, 96%, 97%, 98%, 99%, or 100% identical to the amino acid sequence set forth in SEQ ID NO:9; and (ii) a light chain comprising or consisting of an amino acid sequence that is at least 85%, 90%, 91%, 92%, 93%, 94%, 95%, 96%, 97%, 98%, 99%, or 100% identical to the amino acid sequence set forth in SEQ ID NO:10. In some embodiments, the anti-αvβ6 antibodies selectively bind to αvβ6. In some embodiments, these anti-αvβ6 antibodies or αvβ6-binding fragments thereof block the binding of αvβ6 to its ligand, latency associated peptide (LAP), as determined by blocking of ligand binding either to purified hsαvβ6 or to β6-expressing cells. In some embodiments, these anti-αvβ6 antibody or αvβ6-binding fragments thereof have one or more (e.g., one, two, three, four) of these properties: (i) specifically bind with high affinity to αvβ6; (ii) inhibit the binding of αvβ6 to LAP, fibronectin, vitronectin, or tenascin with an IC50 value lower than that of the 10D5 antibody (WO 99/07405); (iii) block or inhibit activation of TGF-0; (iv) specifically bind to the β6 subunit; and (v) recognize αvβ6 in immunostaining procedures such as immunostaining of paraffin-embedded tissues.

In certain embodiments, these compositions are high concentration anti-αvβ6 antibody compositions. By “high concentration anti-αvβ6 antibody composition” is meant a composition comprising anti-αvβ6 antibodies or αvβ6-binding fragments thereof at a concentration of greater than 100 mg/ml and less than 300 mg/ml. In certain instances, the anti-αvβ6 antibody composition comprises anti-αvβ6 antibodies or αvβ6-binding fragments thereof at a concentration of 50 mg/ml to 250 mg/ml. In certain instances, the anti-αvβ6 antibody composition comprises anti-αvβ6 antibodies or αvβ6-binding fragments thereof at a concentration of 50 mg/ml to 225 mg/ml. In other instances, the anti-αvβ6 antibody composition comprises anti-αvβ6 antibodies or αvβ6-binding fragments thereof at a concentration of 75 mg/ml to 225 mg/ml. In certain instances, the anti-αvβ6 antibody composition comprises anti-αvβ6 antibodies or αvβ6-binding fragments thereof at a concentration of 50 mg/ml to 200 mg/ml. In other instances, the anti-αvβ6 antibody composition comprises anti-αvβ6 antibodies or αvβ6-binding fragments thereof at a concentration of 75 mg/ml to 165 mg/ml. In other instances, the anti-αvβ6 antibody composition comprises anti-αvβ6 antibodies or αvβ6-binding fragments thereof at a concentration of 100 mg/ml to 225 mg/ml. In yet other instances, the anti-αvβ6 antibody composition comprises anti-αvβ6 antibodies or αvβ6-binding fragments thereof at a concentration of 125 mg/ml to 225 mg/ml. In other instances, the anti-αvβ6 antibody composition comprises anti-αvβ6 antibodies or αvβ6-binding fragments thereof at a concentration of 125 mg/ml to 175 mg/ml. In certain instances, the anti-αvβ6 antibody composition comprises anti-αvβ6 antibodies or αvβ6-binding fragments thereof at a concentration of 240 mg/ml. In certain instances, the anti-αvβ6 antibody composition comprises anti-αvβ6 antibodies or αvβ6-binding fragments thereof at a concentration of 225 mg/ml. In certain instances, the anti-αvβ6 antibody composition comprises anti-αvβ6 antibodies or αvβ6-binding fragments thereof at a concentration of 200 mg/ml. In certain instances, the anti-αvβ6 antibody composition comprises anti-αvβ6 antibodies or αvβ6-binding fragments thereof at a concentration of 175 mg/ml. In certain instances, the anti-αvβ6 antibody composition comprises anti-αvβ6 antibodies or αvβ6-binding fragments thereof at a concentration of 150 mg/ml. In other instances, the anti-αvβ6 antibody composition comprises anti-αvβ6 antibodies or αvβ6-binding fragments thereof at a concentration of 125 mg/ml. In some instances, the anti-αvβ6 antibody composition comprises anti-αvβ6 antibodies or αvβ6-binding fragments thereof at a concentration of 100 mg/ml.

A composition (e.g., a pharmaceutical composition) comprising an anti-αvβ6 antibody or αvβ6-binding fragment thereof described herein may be in any one of a variety of forms. These include, for example, liquid solutions (e.g., injectable and infusible solutions), dispersions, or suspensions. The preferred form can depend on the intended mode of administration and therapeutic application. In certain embodiments, a pharmaceutical composition described herein is in the form of a sterile injectable or infusible solution.

Sterile injectable solutions can be prepared by incorporating an antibody described herein in the required amount with one or a combination of ingredients, followed by filtered sterilization. Generally, dispersions are prepared by incorporating an antibody described herein into a sterile vehicle that contains a basic dispersion medium and the required other ingredients. In the case of sterile powders for the preparation of sterile injectable solutions, an exemplary method of preparation is vacuum drying and freeze drying that yields a powder of an antibody described herein plus any additional desired ingredient from a previously sterile-filtered solution thereof. The proper fluidity of a solution can be maintained, for example, by the use of a coating such as lecithin, by the maintenance of the required particle size in the case of dispersion, and by the use of surfactants.

The anti-αvβ6 antibody compositions (e.g., pharmaceutical compositions) may additionally comprise one or more excipients.

In one embodiment, the excipient lowers/reduces the aggregation and/or viscosity of the antibody in the composition compared to aggregation and/or viscosity of the antibody in the pharmaceutical composition without that excipient. In certain embodiments, such an excipient is arginine. In one instance, the excipient is L-arginine hydrochloride. Arginine (e.g., L-arginine hydrochloride) can be included in the composition at a concentration of 40 mM to 260 mM, 50 mM to 250 mM, 50 mM to 200 mM, 50 mM to 150 mM, 50 mM to 125 mM, 50 mM to 100 mM, 75 mM to 250 mM, 75 mM to 200 mM, 75 mM to 150 mM, or 75 mM to 100 mM. In certain embodiments arginine (e.g., Arg.HCl) is present in the composition at a concentration of 50 mM to 250 mM. In other embodiments, arginine (e.g., Arg.HCl) is present in the composition at a concentration of 50 mM to 200 mM. In certain instances, arginine (e.g., arginine hydrochloride) can be included in the composition at a concentration of 80 mM, 100 mM, 120 mM, 125 mM, 130 mM, 135 mM, 140 mM, 145 mM, 150 mM, 220 mM, or 260 mM. In a specific instance, arginine (e.g., arginine hydrochloride) can be included in the composition at a concentration of 100 mM. In another specific instance, arginine (e.g., arginine hydrochloride) can be included in the composition at a concentration of 150 mM.

Sometimes, solutions containing arginine develop visible particles after incubation at room temperature or higher temperatures (e.g., 40° C.). Addition of sucrose can reduce or prevent the formation of visible particles. Furthermore, sucrose can lower the counts of sub visible particulates. In some embodiments, the anti-αvβ6 antibody composition comprises sucrose at a concentration of 0.05% to 5%, 0.05% to 4%, 0.05% to 3%, 1% to 5%, 1% to 4%, 1% to 3%, 2% to 5%, 2% to 4%, or 2% to 3%. In certain embodiments, the anti-αvβ6 antibody composition comprises sucrose at a concentration of 0.5%, 1%, 1.5%, 2%, 2.5%, 3%, 3.5%, 4%, 4.5%, or 5%. In a particular embodiment, the anti-αvβ6 antibody composition comprises sucrose at a concentration of 3%. In another particular embodiment, the anti-αvβ6 antibody composition comprises sucrose at a concentration of 1%.

In one embodiment, the anti-αvβ6 antibody compositions comprise methionine. In one instance, methionine is included in the composition at a concentration from 0.5 mM to 25 mM. In another instance, methionine is included in the composition at a concentration from 1 mM to 10 mM. In another instance, methionine is included in the composition at a concentration from 3 mM to 8 mM. In one instance, methionine is included in the composition at a concentration of 1 mM, 2 mM, 5 mM, 10 mM, 15 mM, 20 mM or 25 mM. In a particular instance, methionine is included in the composition at a concentration of 10 mM. In another particular instance, methionine is included in the composition at a concentration of 5 mM.

Antibody product manufacturing is a complex process that can involve several steps such as, e.g., drug substance and bulk formulation, filtration, shipping, pooling, filling, lyophilization, inspections, packaging, and storage. During these steps, antibodies may be subjected to many different forms of stresses, e.g., agitation, temperature, light exposure, and oxidation. These types of stresses can lead to denaturation and aggregation of the antibody, which compromise the product quality and can even lead to loss of a production batch. Agitation is one of the common physical stresses that antibody therapeutics are subjected to during the course of the manufacturing process. Agitation occurs, e.g., during mixing, ultrafiltration/diafiltration, pumping, shipping, and filling. To protect the antibody composition against agitation-induced stress, the composition may include a polysorbate. In certain embodiments, the composition comprises polysorbate-80 at a concentration of 0.01% to 0.5%, 0.01% to 0.1%, 0.01% to 0.09%, 0.01% to 0.08%, 0.01% to 0.07%, 0.01% to 0.06%, 0.01% to 0.05%, 0.01% to 0.04%, or 0.01% to 0.03%. In certain embodiments, the composition comprises polysorbate-80 at a concentration of 0.02% to 0.08%. In some embodiments, the composition comprises polysorbate-80 at a concentration of 0.01%, 0.02%, 0.03%, 0.04%, 0.05%, 0.06%, 0.07%, 0.08%, 0.09%, or 0.1%. In a particular embodiment, the composition comprises polysorbate-80 at a concentration of 0.05%.

Any antibody composition benefits from a buffer that provides good buffering capacity. In certain embodiments, the antibody composition comprises sodium citrate and citric acid as the buffering agent. In certain embodiments, the composition comprises sodium citrate and citric acid at a concentration of 5 mM to 50 mM, 5 mM to 40 mM, 5 mM to 35 mM, 5 mM to 30 mM, 5 mM to 25 mM, 10 mM to 50 mM, 10 mM to 40 mM, 10 mM to 30 mM, 10 mM to 25 mM, 15 mM to 50 mM, 15 mM to 40 mM, 15 mM to 30 mM, or 15 mM to 25 mM. In certain embodiments, the composition comprises sodium citrate and citric acid at a concentration of 5 mM to 35 mM. In certain embodiments, the composition comprises sodium citrate and citric acid at a concentration of 10 mM to 30 mM. In some embodiments, the composition comprises sodium citrate and citric acid at a concentration of 5 mM, 10 mM, 15 mM, 20 mM, 25 mM, 30 mM, or 35 mM. In a particular embodiment, the composition comprises sodium citrate and citric acid at a concentration of 20 mM.

The pH of the antibody composition can be from 5.0 to 6.5. In certain cases, the pH of the antibody composition can be 5.2 to 6.2. In certain instances, the pH of the antibody composition is 5.0, 5.1, 5.2, 5.3, 5.4, 5.5, 5.6, 5.7, 5.8, 5.9, 6.0, 6.1, 6.2, 6.3, 6.4, or 6.5. In a particular embodiment, the pH of the antibody composition is 5.5.

In some instances, the anti-αvβ6 antibody compositions comprise L-arginine hydrochloride (e.g., 150 mM) and methionine (e.g., 5 mM). In certain cases, these compositions have a pH of 5.5.

In some instances, the anti-αvβ6 antibody compositions comprise L-arginine hydrochloride (e.g., 150 mM), methionine (e.g., 5 mM) and a buffer (e.g., sodium citrate and citric acid at 20 mM). In certain cases, these compositions have a pH of 5.5.

In some instances, the anti-αvβ6 antibody compositions comprise L-arginine hydrochloride (e.g., 150 mM), methionine (e.g., 5 mM), and PS80 (e.g., 0.05%). In certain cases, these compositions have a pH of 5.5.

In certain embodiments, the anti-αvβ6 antibody compositions comprise L-arginine hydrochloride (e.g., 150 mM), methionine (e.g., 5 mM), sodium citrate and citric acid (e.g., 20 mM), and PS80 (e.g., 0.05%), and has a pH of 5.2 to 6.2. In some embodiments, the anti-αvβ6 compositions comprise L-arginine hydrochloride (e.g., 150 mM), methionine (e.g., 5 mM), sodium citrate and citric acid (e.g., 20 mM), and PS80 (e.g., 0.05%), and has a pH of 5.5. In certain embodiments, the anti-αvβ6 compositions comprise L-arginine hydrochloride (e.g., 150 mM), methionine (e.g., 5 mM), sodium citrate and citric acid (e.g., 20 mM), PS80 (e.g., 0.05%), and sucrose (up to 3%), and has a pH of 5.2 to 6.2. In some embodiments, the anti-αvβ6 compositions comprise L-arginine hydrochloride, methionine, sodium citrate and citric acid, PS80, and has a pH of 5.5. In all of these embodiments, the anti-αvβ6 antibody is present at a concentration of 100 mg/ml to 165 mg/ml. In one instance, the anti-αvβ6 antibody is present at a concentration of 150 mg/ml. In one instance, the anti-αvβ6 antibody is present at a concentration of 100 mg/ml.

In some cases, the anti-αvβ6 composition comprises a thiol-containing antioxidant (e.g., reduced glutathione (GSH), oxidized glutathione (GSSG), GSH+GSSG, cysteine, cystine, cysteine+cystine) at a concentration of 0.02 mM to 2 mM (e.g., 0.02, 0.03, 0.05, 0.06, 0.08, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mM). Such thiol-containing antioxidants can cleave unfavorable or misbridged disulfide bonds and promote the formation of favorable or properly bridged disulfide bonds. This would result in the stabilization of the native confirmation of the antibody or fragment thereof and slow down aggregation rates. The antioxidant properties of these molecules may slow down oxidative processes that lead to aggregation. In some cases, the composition comprises GSH at a concentration of 0.4 mM. In some cases, the composition comprises GSSG at a concentration of 0.2 mM. In some cases, the composition comprises GSH at a concentration of 0.4 mM and GSSG at a concentration of 0.2 mM. In some cases, the composition comprises cysteine at a concentration of 0.4 mM. In some cases, the composition comprises cystine at a concentration of 0.2 mM. In some cases, the composition comprises cysteine at a concentration of 0.4 mM and cystine at a concentration of 0.2 mM.

In certain embodiments, the anti-αvβ6 antibody compositions comprise L-arginine hydrochloride (e.g., 150 mM), methionine (e.g., 5 mM), sodium citrate and citric acid (e.g., 20 mM), a thiol-containing antioxidant such as GSH, GSSG, GSH and GSSG, cysteine, cystine, or cysteine and cystine (e.g., 0.02 mM to 2 mM), and PS80 (e.g., 0.05%), and has a pH of 5.2 to 6.2. In some embodiments, the anti-αvβ6 antibody compositions comprise L-arginine hydrochloride (e.g., 150 mM), methionine (e.g., 5 mM), sodium citrate and citric acid (e.g., 20 mM), a thiol-containing antioxidant such as GSH, GSSG, GSH and GSSG, cysteine, cystine, or cysteine and cystine (e.g., 0.02 mM to 2 mM), and PS80 (e.g., 0.05%), and has a pH of 5.5. In certain embodiments, the anti-αvβ6 antibody compositions comprise L-arginine hydrochloride (e.g., 150 mM), methionine (e.g., 5 mM), sodium citrate and citric acid (e.g., 20 mM), PS80 (e.g., 0.05%), a thiol-containing antioxidant such as GSH, GSSG, GSH and GSSG, cysteine, cystine, or cysteine and cystine (e.g., 0.02 mM to 2 mM), and sucrose (up to 3%), and has a pH of 5.2 to 6.2. In some embodiments, the anti-αvβ6 antibody compositions comprise L-arginine hydrochloride, methionine, histidine, PS80, and a thiol-containing antioxidant such as GSH, GSSG, GSH and GSSG, cysteine, cystine, or cysteine and cystine, and has a pH of 5.5. In all of these embodiments, the anti-αvβ6 antibody is present at a concentration of 100 mg/ml to 165 mg/ml. In one instance, the anti-αvβ6 antibody is present at a concentration of 150 mg/ml. In one instance, the anti-αvβ6 antibody is present at a concentration of 100 mg/ml.

In certain embodiments, the anti-αvβ6 antibody compositions comprise L-arginine hydrochloride (e.g., 150 mM), sodium citrate buffer (sodium citrate and citric acid) (e.g., 20 mM), a thiol-containing antioxidant such as GSH, GSSG, GSH and GSSG, cysteine, cystine, or cysteine and cystine (e.g., 0.02 mM to 2 mM), and PS80 (e.g., 0.05%), and has a pH of 5.2 to 6.2. In some embodiments, the anti-αvβ6 antibody compositions comprise L-arginine hydrochloride (e.g., 150 mM), sodium citrate and citric acid (e.g., 20 mM), a thiol-containing antioxidant such as GSH, GSSG, GSH and GSSG, cysteine, cystine, or cysteine and cystine (e.g., 0.02 mM to 2 mM), and PS80 (e.g., 0.05%), and has a pH of 5.5. In certain embodiments, the anti-αvβ6 antibody compositions comprise L-arginine hydrochloride (e.g., 150 mM), sodium citrate and citric acid (e.g., 20 mM), PS80 (e.g., 0.05%), a thiol-containing antioxidant such as GSH, GSSG, GSH and GSSG, cysteine, cystine, or cysteine and cystine (e.g., 0.02 mM to 2 mM), and sucrose (up to 3%), and has a pH of 5.2 to 6.2. In some embodiments, the anti-αvβ6 antibody compositions comprise L-arginine hydrochloride, histidine, PS80, and a thiol-containing antioxidant such as GSH, GSSG, GSH and GSSG, cysteine, cystine, or cysteine and cystine, and has a pH of 5.5. In all of these embodiments, the anti-αvβ6 antibody is present at a concentration of 100 mg/ml to 165 mg/ml. In one instance, the anti-αvβ6 antibody is present at a concentration of 150 mg/ml. In one instance, the anti-αvβ6 antibody is present at a concentration of 100 mg/ml.

In certain embodiments, the composition (e.g., a pharmaceutical composition) comprises an anti-αvβ6 antibody or an αvβ6-binding fragment thereof at a concentration of 75 mg/ml to 250 mg/ml, arginine (e.g., L-arginine hydrochloride) at a concentration of 50 mM to 200 mM, methionine at a concentration of 1 mM to 10 mM; polysorbate-80 at a concentration of 0.01% to 0.1%, sodium citrate and citric acid at a concentration of 10 mM to 30 mM, and sucrose at a concentration of 0% to 3%. The composition has a pH of 5.2 to 6.0. In certain embodiments, the anti-αvβ6 antibody or an αvβ6-binding fragment thereof of the composition comprises a VH and a VL comprising the CDRs of STX-100 (e.g., SEQ ID NOs: 1 or 11, 2, 3, 4, 5, and 6). In certain embodiments, the anti-αvβ6 antibody or an αvβ6-binding fragment thereof of the composition comprises a VH and a VL comprising SEQ ID NOs: 7 and 8, respectively. In some embodiments, the anti-αvβ6 antibody or an αvβ6-binding fragment thereof of the composition comprises a heavy chain and a light chain comprising SEQ ID NOs: 9 and 10, respectively. In one embodiment, the composition has a pH of 5.5 and comprises STX-100 or a STX-100-binding fragment thereof at a concentration of 150 mg/ml, L-arginine hydrochloride at a concentration of 150 mM, methionine at a concentration of 5 mM, polysorbate-80 at a concentration of 0.05%, and sodium citrate and citric acid at a concentration of 20 mM. In certain embodiments, the composition further comprises a thiol-containing antioxidant (e.g., GSH, GSSG, GSH+GSSG, cysteine, cystine, cysteine+cystine) at a concentration of 0.02 mM to 2 mM. In some embodiments, the composition further comprises sucrose at a concentration of 0.01% to 3%. In certain embodiments, the anti-αvβ6 antibody or an αvβ6-binding fragment thereof of the composition comprises a VH and a VL comprising the CDRs of STX-100 (e.g., SEQ ID NOs: 1 or 11, 2, 3, 4, 5, and 6). In certain embodiments, the anti-αvβ6 antibody or an αvβ6-binding fragment thereof of the composition comprises a VH and a VL comprising SEQ ID NOs: 7 and 8, respectively. In some embodiments, the anti-αvβ6 antibody or an αvβ6-binding fragment thereof of the composition comprises a heavy chain and a light chain comprising SEQ ID NOs: 9 and 10, respectively.

In one embodiment, the composition has a pH of 5.5 and comprises STX-100 or a STX-100-binding fragment thereof at a concentration of 150 mg/ml, L-arginine hydrochloride at a concentration of 150 mM, a thiol-containing antioxidant (e.g., GSH, GSSG, GSH+GSSG, cysteine, cystine, cysteine+cystine) at a concentration of 0.02 mM to 2 mM, polysorbate-80 at a concentration of 0.05%, and sodium citrate and citric acid at a concentration of 20 mM. In one embodiment, the thiol-containing antioxidant is GSH at a concentration of 0.4 mM. In one embodiment, the thiol-containing antioxidant is GSH at a concentration of 0.4 mM and GSSG at a concentration of 0.2 mM. In another embodiment, the thiol-containing antioxidant is cysteine at a concentration of 0.4 mM. In another embodiment, the thiol-containing antioxidant is cysteine at a concentration of 0.4 mM and cystine at a concentration of 0.2 mM.

### Dosing

The anti-αvβ6 antibody (e.g., STX-100) or αvβ6-binding fragment thereof described above can be administered to a subject, e.g., a human subject, at different doses. The anti-αvβ6 antibody (e.g., STX-100) or αvβ6-binding fragment thereof can be administered as a fixed dose (i.e., independent of the weight of the patient), or in a mg/kg dose (i.e., a dose which varies based on the weight of the subject). Dosage unit form or “fixed dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent. Single or multiple dosages may be given. The treatment can continue for days, weeks, months or even years.

In certain embodiments, for treating an indication described herein in an adult human subject, the dosage of the anti-αvβ6 antibody (e.g., STX-100) or αvβ6-binding fragment thereof is a fixed dose of 40 mg to 64 mg once weekly. In one embodiment, for treating an indication described herein in an adult human subject, the dosage of the anti-αvβ6 antibody (e.g., STX-100) or αvβ6-binding fragment thereof is a fixed dose of 40 mg once weekly. In another embodiment, the dosage of the anti-αvβ6 antibody or αvβ6-binding fragment thereof is a fixed dose of 48 mg once weekly. In another embodiment, the dosage of the anti-αvβ6 antibody or αvβ6-binding fragment thereof is a fixed dose of 56 mg once weekly. In another embodiment, the dosage of the anti-αvβ6 antibody or αvβ6-binding fragment thereof is a fixed dose of 64 mg once weekly.

In certain embodiments, for treating an indication described herein in an adult human subject, the dosage of the anti-αvβ6 antibody (e.g., STX-100) or αvβ6-binding fragment thereof is a mg/kg dose of 0.3 mg/kg to 1.0 mg/kg. In one embodiment, for treating an indication described herein in an adult human subject, the dosage of the anti-αvβ6 antibody (e.g., STX-100) or αvβ6-binding fragment thereof is a mg/kg dose of 0.5 mg/kg to 0.8 mg/kg. In one embodiment, for treating an indication described herein in an adult human subject, the dosage of the anti-αvβ6 antibody (e.g., STX-100) or αvβ6-binding fragment thereof is a mg/kg dose of 0.5 mg/kg. In another embodiment, for treating an indication described herein in an adult human subject, the dosage of the anti-αvβ6 antibody (e.g., STX-100) or αvβ6-binding fragment thereof is a mg/kg dose of 0.6 mg/kg. In another embodiment, for treating an indication described herein in an adult human subject, the dosage of the anti-αvβ6 antibody (e.g., STX-100) or αvβ6-binding fragment thereof is a mg/kg dose of 0.7 mg/kg. In yet another embodiment, for treating an indication described herein in an adult human subject, the dosage of the anti-αvβ6 antibody (e.g., STX-100) or αvβ6-binding fragment thereof is a mg/kg dose of 0.8 mg/kg.

In certain instances, the anti-αvβ6 antibody or αvβ6-binding fragment thereof is administered in combination with a therapeutically effective amount of an art recognized treatment for IPF.

Exemplary art recognized treatment options that can be used in combination with the antibody of the invention include: Corticosteroids (prednisone); Cyclophosphamide (Cytoxan®); Azathioprine (Imuran®); Mycophenolate mofetil (Cellcept®, Myfortic®); N-acetylcysteine (NAC); Nintedanib (Ofev®); Pirfenidone (Esbriet®, Pirfenex®, Pirespa®); Proton pump inhibitors (Prilosec OTC®, Nexium®, others); or Supplemental Oxygen Therapy.

In one embodiment, an antibody of the invention is combined with prifenidone or nintedanib. In certain cases, the subject is administered prifenidone as follows:

Treatment Days Dosage

Days 1 through 7 267 mg three times daily (801 mg/day)

Days 8 through 14 534 mg three times daily (1602 mg/day)

Days 15 onward 801 mg three times daily (2403 mg/day)

in combination with the antibody of the invention. In certain cases, the subject is administered a therapeutically effective amount of nintedanib at a fixed dose of 150 mg twice daily in combination with the antibody of the invention.

In certain instances, the anti-αvβ6 antibody or αvβ6-binding fragment thereof is administered in combination with an antibody that inhibits the activity of connective tissue growth factor (CTGF) such as, but not limited to, the fully-human monoclonal antibody, Pamrevlumab.

In certain instances, the anti-αvβ6 antibody or αvβ6-binding fragment thereof is administered in combination with a therapeutically effective amount of a selective autotaxin inhibitor (e.g., GLPG1690).

In certain instances, the anti-αvβ6 antibody or αvβ6-binding fragment thereof is administered in combination with a therapeutically effective amount of GBT-440.

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual. A therapeutically effective amount is also one in which any toxic, or detrimental effects, of the composition is outweighed by the therapeutically beneficial effects. In certain embodiment, the therapeutically effective amount of the anti-αvβ6 antibody or αvβ6-binding fragment thereof is 40 mg to 64 mg. In one embodiment, the therapeutically effective amount of the anti-αvβ6 antibody or αvβ6-binding fragment thereof is 40 mg. In another embodiment, the therapeutically effective amount of the anti-αvβ6 antibody or αvβ6-binding fragment thereof is 48 mg. In yet another embodiment, the therapeutically effective amount of the anti-αvβ6 antibody or αvβ6-binding fragment thereof is 56 mg. In yet another embodiment, the therapeutically effective amount of the anti-αvβ6 antibody or αvβ6-binding fragment thereof is 64 mg.

The route and/or mode of administration of the anti-αvβ6 antibody or αvβ6-binding fragment thereof can be tailored for the individual subject. For many applications, the route of administration is one of: subcutaneous injection (SC), intravenous injection or infusion (IV), intraperitoneal administration (IP), or intramuscular injection. In one embodiment, the route of administration is subcutaneous. In another embodiment, the route of administration is intravenous.

Pharmaceutical compositions that comprise the anti-αvβ6 antibody or αvβ6-binding fragment thereof alone or in combination with non αvβ6 antibody agent(s) can be administered with a medical device. The device can be designed with features such as portability, room temperature storage, and ease of use so that it can be used in emergency situations, e.g., by an untrained subject or by emergency personnel in the field, removed to medical facilities and other medical equipment. The device can include, e.g., one or more housings for storing pharmaceutical preparations that include the anti-αvβ6 antibody or αvβ6-binding fragment thereof, and can be configured to deliver one or more unit doses of the anti-αvβ6 antibody or other agent.

For example, the pharmaceutical composition can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other devices, implants, delivery systems, and modules are also known.

In one embodiment, the anti-αvβ6 antibody or αvβ6-binding fragment thereof is administered to a human subject with a syringe. In another embodiment, the anti-αvβ6 antibody or αvβ6-binding fragment thereof is administered to a human subject with a pump for subcutaneous delivery. In some embodiments, the anti-αvβ6 antibody or αvβ6-binding fragment thereof is administered to a human subject with an autoinjector. In other embodiments, the anti-αvβ6 antibody or αvβ6-binding fragment thereof is administered to a human subject with a subcutaneous large volume injector.

This disclosure provides a pump or syringe comprising a sterile preparation of an anti-αvβ6 antibody (e.g., STX-100) or αvβ6-binding fragment thereof. The syringe or pump can be adapted for subcutaneous administration of the anti-αvβ6 antibody or αvβ6-binding fragment thereof. In some cases, the syringe or pump delivers a fixed doses(s) (e.g., 40 mg, 48 mg, 56 mg, 64 mg) of the anti-αvβ6 antibody or αvβ6-binding fragment thereof.

The disclosure also provides a pump, syringe, or injector (e.g., autoinjector, subcutaneous large volume injector) comprising a sterile preparation of the pharmaceutical compositions described above. The syringe or pump can be adapted for subcutaneous administration of the pharmaceutical compositions comprising the anti-αvβ6 antibody or αvβ6-binding fragment thereof. In some instances, the syringe or pump delivers a fixed doses(s) (e.g., 40 mg, 48 mg, 56 mg, 64 mg) of the anti-αvβ6 antibody or αvβ6-binding fragment thereof

### Methods of Treatment

The antibodies of this disclosure are useful in the treatment, including prevention, of αvβ6-mediated diseases. For example, these antibodies can be used to treat fibrosis (e.g., lung fibrosis, kidney fibrosis, liver fibrosis, cardiac fibrosis), acute lung injury, acute kidney injury, Alport's Syndrome, psoriasis, scleroderma, and sclerosis of lung, liver, or kidney, by blocking the activation of TGF-β or blocking the binding of β6 to any other ligands, such as fibronectin, vitronectin, and tenascin. The novelty of this approach includes: (1) it blocks the activation of TGF-β rather than the binding of TGF-β to its receptor, (2) it can inhibit TGF-β locally (i.e., at sites of αvβ6 upregulation) rather than systemically, and (3) it inhibits binding of αvβ6 to a ligand.

Other than fibrotic diseases or conditions, the antibodies of the disclosure are useful in treating cancer or cancer metastasis (including tumor growth and invasion), particularly epithelial cancers. A subset of epithelial cancers is squamous cell carcinoma, e.g., head and neck, oral, breast, lung, prostate, cervical, pharyngeal, colon, pancreatic and ovarian cancers.

In additional embodiments of the invention, αvβ6-binding antibodies or fragments thereof, may be used in therapeutic regimens for treating humans having, or at risk of developing carcinomas. Such methods of the invention are useful in treating cancer and associated events, including tumor growth, metastasis and angiogenesis. Particularly amenable to such an approach are those diseases or cancers that are characterized by increased levels of αvβ6 expression in the tissues or cells of a mammal suffering from the disease, and which are responsive to treatments, which target the tissues or cells expressing increased levels of αvβ6 and eliminate those tissues or cells. Diseases that are particularly treatable by these methods include metastatic cancers of epithelial tissues (i.e., metastatic carcinomas and/or adenocarcinomas), including of the breast, ovary, prostate, liver, lung, pancreas, colon, head and neck tissues (e.g., oral, pharyngeal, lingual and laryngeal tissues), endometrium, cervix, stomach and spleen. Particularly suitable for treatment by these methods of the present invention are carcinomas of the endometrium, pancreas, colon (e.g., colorectal carcinomas), cervix, lung and breast (including ductal carcinoma in situ (DCIS) and lobular carcinoma in situ (LCIS) of the breast).

The following are examples of the practice of the invention. They are not to be construed as limiting the scope of the invention in any way.

### EXAMPLES

These Examples relate, in part, to the development of a stable high concentration (e.g., 100 mg/ml or greater) liquid formulation for STX-100.

### Example 1: Pre-Formulation Evaluation

During the initial pre-formulation evaluation, accelerated stability studies were conducted to explore pH, buffer, and excipient components suitable for a high concentration liquid formulation for STX-100. A formulation matrix containing 10 mM Na-citrate/citric acid pH 5.0, 150 mM arginine hydrochloride (Arginine-HCl) was used as a control for comparison. For the excipient screen, amino acids like glycine, lysine, arginine-HCl, and methionine, sugars like sorbitol, trehalose, mannitol, sucrose, and buffer systems such as citrate and acetate were tested. A pH range from 4.4 to 5.7 was also evaluated.

Accelerated stability evaluation was performed at 40° C. incubation over 4 weeks for the formulations. The following quality attributes were monitored: visible particulates and clarity, % high molecular weight species (via SEC), total sub-visible particulates (via MFI), turbidity (via OD340), pH, fragmentation (via GXII), % total acidic isoforms (via iCIEF), and viscosity at TO. Maximum weightage was assigned to Critical quality attributes (CQA) like aggregate level and particle formation and were utilized in formulation selection. The formulations with least amount of aggregate level and particle formation were selected for further evaluations.

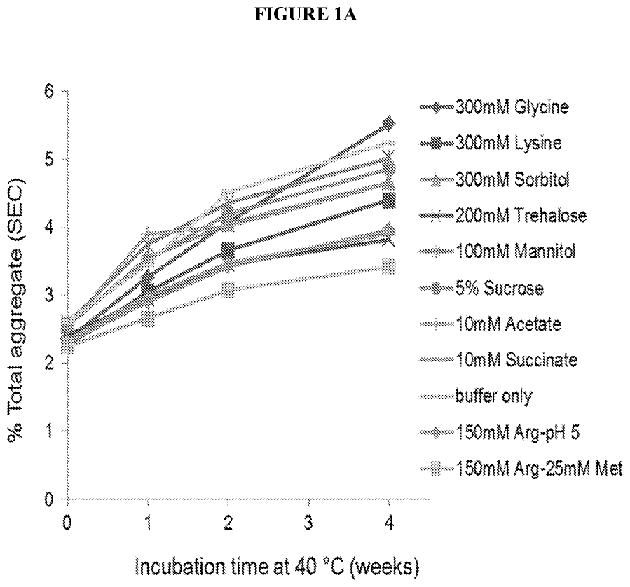

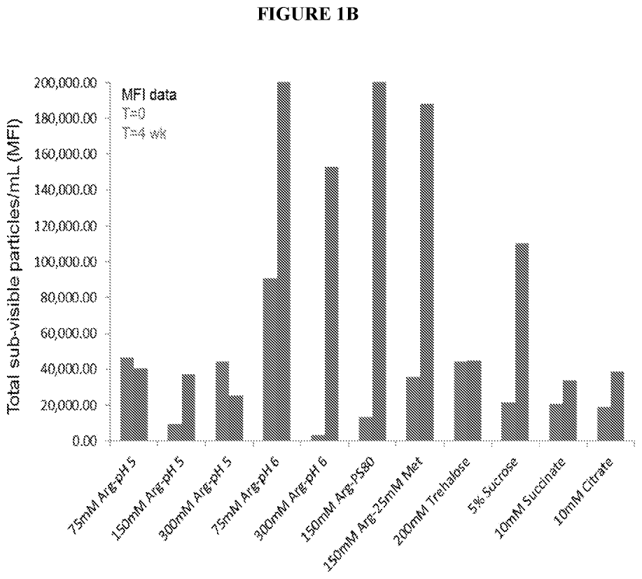

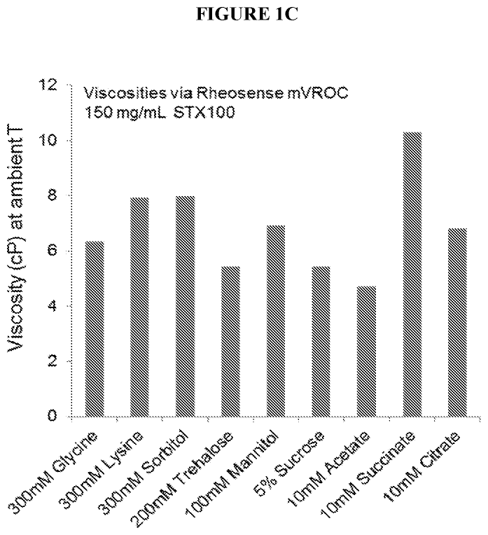

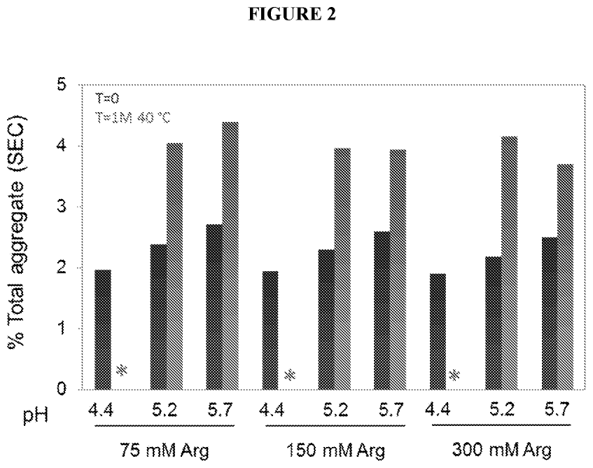

The data indicated that arginine- and trehalose-containing formulations to be the most stable compared to others (Table 2; FIGS. 1A-1C; and FIG. 2).

TABLE 2
Results of pre-formulation buffer-excipient screening study

| | Visible appearance | | | | Turbidity (OD340) | | | |
|---|---|---|---|---|---|---|---|---|
| | | Time at 40° C. | | | | Time at 40° C. | | |
| Excipient/buffer | T = 0 | 1 wk | 2 wk | 4 wk | T = 0 | 1 wk | 2 wk | 4 wk |
| 300 mM Glycine | 5-10 | 10-50 | <10 | <10 | 1.591549 | 1.0812 | 1.193952 | NT |
| | particles | particles | particles | particles | | | | |
| | NT | cla: 18- | cla: 18- | cla: 30- | | | | |
| | | 30 NTU | 30 NTU | 50 NTU | | | | |
| 300 mM Lysine | 5-10 | 10-50 | Few | <10 | 0.916307 | 1.1386 | 1.30471 | NT |
| | particles | particles | white | particles | | | | |
| | | | particles | | | | | |
| | NT | cla: 18- | cla: 18- | cla: 30- | | | | |
| | | 30 NTU | 30 NTU | 50 NTU | | | | |
| 300 mM Sorbitol | 50-100 | <10 | <10 | <10 | 0.85723 | 1.1036 | 1.161319 | NT |
| | particles | particles | particles | particles | | | | |
| | NT | cla: 18- | cla: 18- | cla: 30- | | | | |
| | | 30 NTU | 30 NTU | 50 NTU | | | | |
| 200 mM Trehalose | Few | <10 | NT | <10 | 1.004 | 1.005 | 1.048 | NT |
| | particles | particles | | particles | | | | |
| | observed | | | | | | | |
| | NT | cla: 18- | NT | cla: 30- | | | | |
| | | 30 NTU | | 50 NTU | | | | |
| 100 mM Mannitol | 5-10 | 10-50 | <10 | <10 | 0.877126 | 1.1184 | 1.181954 | NT |
| | particles | particles | particles | particles | | | | |
| | NT | cla: 18- | cla: 18- | cla: 30- | | | | |
| | | 30 NTU | 30 NTU | 50 NTU | | | | |
| 5% Sucrose | Few | <10 | <10 | <10 | 1.902725 | 1.1286 | 1.192552 | NT |
| | particles | particles | particles | particles | | | | |
| | observed | | | | | | | |
| | NT | cla: 18- | cla: 18- | cla: 30- | | | | |
| | | 30 NTU | 30 NTU | 50 NTU | | | | |
| 10 mM Acetate | <50 | <10 | <10 | <10 | 0.71461 | 0.963 | 1.0212 | NT |
| | particles | particles | particles | particles | | | | |
| | NT | cla: 18- | cla: 18- | cla: 30- | | | | |
| | | 30 NTU | 30 NTU | 50 NTU | | | | |
| 10 mM Succinate | <50 | <10 | <10 | <10 | 0.925 | 0.923 | 0.929 | NT |
| | particles | particles | particles | particles | | | | |
| | NT | cla: 18- | cla: 18- | cla: 30- | | | | |
| | | 30 NTU | 30 NTU | 50 NTU | | | | |
| 10 mM Citrate | <50 | <10 | <10 | <10 | 0.903355 | 1.1226 | 1.178035 | NT |
| | particles | particles | particles | particles | | | | |
| | NT | cla: 18- | cla: 18- | cla: 30- | | | | |
| | | 30 NTU | 30 NTU | 50 NTU | | | | |

NT: Not tested

The presence of additional 25 mM methionine conferred greater aggregation resistance to the arginine-containing formulation (FIG. 1A).

At pH 4.4, formulations containing 75 mM to 300 mM Arg-HCl displayed gel formation whereas in the pH 5.2 to 5.7 range, no gel formation was observed under accelerated conditions (FIG. 2). As seen in FIGS. 1A and 2, the % total aggregate increase observed in these formulations was also lower in the entire buffer-excipient screen and the pH-arginine screen. At 150 mM ArgHCl, there was no significant difference in aggregation between pH 5.2 and pH 5.7 (FIG. 2) suggesting that this pH range would most likely contain the desired set-point for minimizing aggregation.

### Example 2: Medium to Long-term Stability Study & Formulation Selection

Based on the pre-formulation results in Example 1, the following five liquid formulations and corresponding container-closures (CCs) were selected for pursuing a long-term (24 month) stability study:
1) Lot #18169-62: 150 mg/mL STX-100 in 20 mM Na-citrate/citric acid, pH 5.3, 150 mM arginine hydrochloride (Arg.HCl), 0.05% PS-80 (1 mL fill in 3 mL Schott vial)
2) Lot #18169-64: 150 mg/mL STX-100 in 20 mM Na-citrate/citric acid, pH 5.3, 150 mM arginine hydrochloride (Arg.HCl), 0.05% PS-80 (1 mL fill in BD Hypak pre-filled syringe, 27 G needle)
3) Lot #18169-66: 150 mg/mL STX-100 in 20 mM Na-citrate/citric acid, pH 5.3, 150 mM arginine hydrochloride (Arg.HCl), 25 mM methionine, 0.05% PS-80 (1 mL fill in BD Hypak pre-filled syringe, 27 G needle)
4) Lot #18169-67: 150 mg/mL STX-100 in 20 mM Na-citrate/citric acid, pH 5.3, 200 mM trehalose, 0.05% PS-80 (1 mL fill in BD Hypak pre-filled syringe, 27 G needle)
5) Lot #18169-72: 250 mg/mL STX-100 in 20 mM Na-citrate/citric acid, pH 5.3, 150 mM arginine hydrochloride (Arg.HCl), 25 mM methionine, 0.05% PS-80 (1 mL fill in BD Hypak pre-filled syringe, 27 G needle)
   Aggregation Data: Stability data indicated that all the five formulations listed above displayed a low aggregation propensity throughout 12 months of storage at 5° C. with only a 0.2-0.3% increase in % total aggregate across all the formulations (Table 3).

TABLE 3
Long-term % Total aggregate data measured using SEC-UPLC

| | Temperature | | | | | | | |
|---|---|---|---|---|---|---|---|---|
| Lot number | (° C.) | % Total aggregates (via SEC-UPLC) | | | | | | |
| Time (months) | | 0 | 1 | 2 | 3 | 6 | 9 | 12 |
| # 18169-62 | 5 | 2.5 | 2.5 | 2.4 | 2.5 | 2.5 | 2.6 | 2.6 |
| 150 mg/mL, 20 mM | 25 | 2.5 | 2.6 | 2.7 | 2.8 | 3.2 | 3.4 | 3.6 |
| citrate, pH 5.3, | 40 | 2.5 | 3.7 | 4.2 | 5.1 | 11.6 | NT | NT |
| 150 mM ArHCl, | | | | | | | | |
| 0.05% PS-80 | | | | | | | | |
| 1 mL in 3 mL | | | | | | | | |
| Schott vial) | | | | | | | | |
| # 18169-64 | 5 | 2.5 | 2.5 | 2.4 | 2.5 | 2.6 | 2.7 | 2.7 |
| 150 mg/mL, 20 mM | 25 | 2.5 | 2.7 | 2.7 | 2.9 | 3.2 | 3.5 | 3.7 |
| citrate, pH 5.3, | 40 | 2.5 | 3.6 | 4.1 | 5 | 11.9 | NT | NT |
| 150 mM ArHCl, | | | | | | | | |
| 0.05% PS-80 | | | | | | | | |
| (1 mL in BD | | | | | | | | |
| Hypak syringe, | | | | | | | | |
| 27 G needle) | | | | | | | | |
| # 18169-66 | 5 | 2.5 | 2.5 | 2.4 | 2.5 | 2.5 | 2.6 | 2.6 |
| 150 mg/mL, 20 mM | 25 | 2.5 | 2.6 | 2.6 | 2.7 | 3 | 3.2 | 3.2 |
| citrate, pH 5.3, | 40 | 2.5 | 3.4 | 3.8 | 4.7 | 11 | NT | NT |
| 150 mM ArHCl, | | | | | | | | |
| 25 mM methionine, | | | | | | | | |
| 0.05% PS-80 | | | | | | | | |
| (1 mL in BD | | | | | | | | |
| Hypak syringe, | | | | | | | | |
| 27 G needle) | | | | | | | | |
| # 18169-67 | 5 | 3 | 3 | 2.9 | 3 | 3.1 | 3.3 | 3.3 |
| 150 mg/mL, 20 mM | 25 | 3 | 3.3 | 3.5 | 3.8 | 4.2 | 4.6 | 4.9 |
| citrate, pH 5.3, | 40 | 3 | 4.5 | 5.3 | 6.2 | 10 | NT | NT |
| 200 mM trehalose, | | | | | | | | |
| 0.05% PS-80 | | | | | | | | |
| (1 mL in BD | | | | | | | | |
| Hypak syringe, | | | | | | | | |
| 27 G needle) | | | | | | | | |
| # 18169-72 | 5 | 1.4 | 1.4 | 1.3 | 1.5 | 1.5 | 1.7 | 1.7 |
| 250 mg/mL, 20 mM | 25 | 1.4 | 1.8 | 1.9 | 2.2 | 2.6 | 3 | 3.2 |
| citrate, pH 5.3, | 40 | 1.4 | 3.1 | 3.9 | 5.2 | 14.9 | NT | NT |
| 150 mM ArHCl, | | | | | | | | |
| 25 mM methionine, | | | | | | | | |
| 0.05% PS-80 | | | | | | | | |
| (1 mL in BD | | | | | | | | |
| Hypak syringe, | | | | | | | | |
| 27 G needle) | | | | | | | | |

The data also indicate that there is no significant difference in aggregate level for formulations held in a pre-filled syringe (18169-64) and a vial (18169-62).
Sub-Visible Particulate (SVP) Data: While the aggregation data was promising for a stable liquid formulation, there were some indications of high sub-visible particulate (SVP) counts via micro-flow imaging, MFI (Tables 4, 5 and 6).

TABLE 4
Total sub-visible particulates/mL (MFI)

| | Temp | | | | | | |
|---|---|---|---|---|---|---|---|
| Lot number | (° C.) | Total Particulates/mL (MFI) | | | | | |
| Time (months) | | 0 | 1 | 3 | 6 | 9 | 12 |
| # 18169-62 | 5 | 13616 | 5005 | 8126 | 40458 | 41396 | 16988 |
| 150 mg/mL, 20 mM | 25 | 13616 | 26252 | 34202 | 311593 | 518112 | 665292 |
| citrate, pH 5.3, | 40 | 13616 | 852037 | 776326 | 2397797 | NT | NT |
| 150 mM ArHCl, | | | | | | | |
| 0.05% PS-80 | | | | | | | |
| 1 mL in 3 mL | | | | | | | |
| Schott vial) | | | | | | | |
| # 18169-64 | 5 | 43413 | 35963 | 15064 | 5777 | 8076 | 8058 |
| 150 mg/mL, 20 mM | 25 | 43413 | 26300 | 68631 | 156553 | 278577 | 208307 |
| citrate, pH 5.3, | 40 | 43413 | 708893 | 1467637 | 2111368 | NT | NT |
| 150 mM ArHCl, | | | | | | | |
| 0.05% PS-80 | | | | | | | |
| (1 mL in BD | | | | | | | |
| Hypak syringe, | | | | | | | |
| 27 G needle) | | | | | | | |
| # 18169-66 | 5 | 19412 | 6536 | 11753 | 13795 | 16704 | 13263 |
| 150 mg/mL, 20 mM | 25 | 19412 | 15750 | 136088 | 236386 | 427144 | 566722 |
| citrate, pH 5.3, | 40 | 19412 | 1230328 | 2023654 | 2003136 | NT | NT |
| 150 mM ArHCl, | | | | | | | |
| 25 mM methionine, | | | | | | | |
| 0.05% PS-80 | | | | | | | |
| (1 mL in BD | | | | | | | |
| Hypak syringe, | | | | | | | |
| 27 G needle) | | | | | | | |
| # 18169-67 | 5 | 25444 | 23998 | 17266 | 27329 | 135928 | 199303 |
| 150 mg/mL, 20 mM | 25 | 25444 | 113162 | 701629 | 527438 | 1602970 | 927081 |
| citrate, pH 5.3, | 40 | 25444 | 178718 | 1183228 | 542746 | NT | NT |
| 200 mM trehalose, | | | | | | | |
| 0.05% PS-80 | | | | | | | |
| (1 mL in BD | | | | | | | |
| Hypak syringe, | | | | | | | |
| 27 G needle) | | | | | | | |
| # 18169-72 | 5 | 13482 | 17008 | 7760 | 10865 | 10633 | 21093 |
| 250 mg/mL, 20 mM | 25 | 13482 | 12741 | 12641 | 18973 | 63098 | 15062 |
| citrate, pH 5.3, | 40 | 13482 | 25198 | 10126 | 21717 | NT | NT |
| 150 mM ArHCl, | | | | | | | |
| 25 mM methionine, | | | | | | | |
| 0.05% PS-80 | | | | | | | |
| (1 mL in BD | | | | | | | |
| Hypak syringe, | | | | | | | |
| 27 G needle) | | | | | | | |
| Ref buffer vial | 5 | 22263 | NT | 86201 | 51685 | NT | 26252 |
| | 25 | 22263 | NT | 57860 | 40409 | NT | 27959 |
| | 40 | 22263 | NT | 56260 | 33898 | NT | NT |
| Ref buffer PFS | 5 | 38840 | NT | 76251 | 64420 | NT | 15914 |
| | 25 | 38840 | NT | 105080 | 40830 | NT | 71146 |
| | 40 | 38840 | NT | 123503 | 236238 | NT | NT |

TABLE 5
Sub-visible particulates (>10 um)/mL (MFI)

| | | Particulates/mL (size > 10 um) | | | | | |
|---|---|---|---|---|---|---|---|
| | | Time (months) | | | | | |
| Lot number | Temp | 0 | 1 | 3 | 6 | 9 | 12 |
| # 18169-62 | 5 | 161 | 94 | 59 | 98 | 374 | 78 |
| 150 mg/mL, 20 mM | 25 | 161 | 128 | 450 | 6376 | 3781 | 10313 |
| citrate, pH 5.3, 150 mM | 40 | 161 | 4019 | 50460 | 57928 | NT | NT |
| ArHCl, 0.05% PS-80 | | | | | | | |
| (1 mL in 3 mL Schott | | | | | | | |
| vial) | | | | | | | |
| # 18169-64 | 5 | 195 | 274 | 300 | 128 | 1934 | 52 |
| 150 mg/mL, 20 mM | 25 | 195 | 872 | 1438 | 6298 | 5127 | 7942 |
| citrate, pH 5.3, 150 mM | 40 | 195 | 20497 | 36305 | 106721 | NT | NT |
| ArHCl, 0.05% PS-80 | | | | | | | |
| (1 mL in BD Hypak | | | | | | | |
| syringe, 27 G needle) | | | | | | | |
| # 18169-66 | 5 | 331 | 70 | 204 | 74 | 1014 | 104 |
| 150 mg/mL, 20 mM | 25 | 331 | 70 | 71 | 7838 | 7696 | 19841 |
| citrate, pH 5.3, 150 mM | 40 | 331 | 32528 | 60842 | 91013 | NT | NT |
| ArHCl, 25 mM | | | | | | | |
| methionine, 0.05% PS-80 | | | | | | | |
| (1 mL in BD Hypak | | | | | | | |
| syringe, 27 G needle) | | | | | | | |
| # 18169-67 | 5 | 243 | 698 | 170 | 292 | 4273 | 2997 |
| 150 mg/mL, 20 mM | 25 | 243 | 3593 | 15312 | 41916 | 52119 | 67621 |
| citrate, pH 5.3, 200 mM | 40 | 243 | 5843 | 35691 | 27479 | NT | NT |
| trehalose, 0.05% PS-80 | | | | | | | |
| (1 mL in BD Hypak | | | | | | | |
| syringe, 27 G needle) | | | | | | | |
| # 18169-72 | 5 | 995 | 3121 | 688 | 1272 | 796 | 870 |
| 250 mg/mL, 20 mM | 25 | 995 | 1112 | 860 | 1036 | 3039 | 308 |
| citrate, pH 5.3, 150 mM | 40 | 995 | 2511 | 786 | 1816 | NT | NT |
| ArgHCl, 25 mM | | | | | | | |
| methionine, 0.05% PS-80 | | | | | | | |
| (1 mL in BD Hypak | | | | | | | |
| syringe, 27 G needle) | | | | | | | |
| Ref buffer vial | 5 | 305 | NT | 852 | 20 | NT | 20 |
| | 25 | 305 | NT | 366 | 64 | NT | 54 |
| | 40 | 305 | NT | 108 | 194 | NT | NT |
| Ref buffer PFS | 5 | 258 | NT | 494 | 370 | NT | 114 |
| | 25 | 258 | NT | 348 | 238 | NT | 420 |
| | 40 | 258 | NT | 822 | 968 | NT | NT |

TABLE 6
Sub-visible particulates (>25 um)/mL (MFI)

| | | Particulates (>25 um); <=600 | | | | | |
|---|---|---|---|---|---|---|---|
| | | Time (months) | | | | | |
| Lot number | Temperature | 0 | 1 | 3 | 6 | 9 | 12 |
| # 18169-62 | 5 | 20 | 22 | 6 | 4 | 16 | 0 |
| 150 mg/mL, 20 mM | 25 | 20 | 8 | 20 | 94 | 66 | 160 |
| citrate, pH 5.3, 150 mM | 40 | 20 | 3123 | 4727 | 679 | NT | NT |
| ArHCl, 0.05% PS-80 | | | | | | | |
| (1 mL in 3 mL Schott | | | | | | | |
| vial) | | | | | | | |
| # 18169-64 | 5 | 27 | 24 | 16 | 2 | 1644 | 0 |
| 150 mg/mL, 20 mM | 25 | 27 | 498 | 132 | 420 | 326 | 640 |
| citrate, pH 5.3, 150 mM | 40 | 27 | 2395 | 2735 | 12288 | NT | NT |
| ArHCl, 0.05% PS-80 | | | | | | | |
| (1 mL in BD Hypak | | | | | | | |
| syringe, 27 G needle) | | | | | | | |
| # 18169-66 | 5 | 117 | 2 | 66 | 6 | 560 | 2 |
| 150 mg/mL, 20 mM | 25 | 179 | 2 | 162 | 560 | 712 | 1570 |
| citrate, pH 5.3, 150 mM | 40 | 117 | 3471 | 6200 | 9676 | NT | NT |
| ArHCl, 25 mM | | | | | | | |
| methionine, 0.05% PS-80 | | | | | | | |
| (1 mL in BD Hypak | | | | | | | |
| syringe, 27 G needle) | | | | | | | |
| # 18169-67 | 5 | 43 | 410 | 42 | 80 | 908 | 428 |
| 150 mg/mL, 20 mM | 25 | 43 | 804 | 2091 | 5815 | 5711 | 8822 |
| citrate, pH 5.3, 200 mM | 40 | 43 | 624 | 5067 | 2915 | NT | NT |
| trehalose, 0.05% PS-80 | | | | | | | |
| (1 mL in BD Hypak | | | | | | | |
| syringe, 27 G needle) | | | | | | | |
| # 18169-72 | 5 | 563 | 2307 | 460 | 850 | 450 | 308 |
| 250 mg/mL, 20 mM | 25 | 563 | 710 | 402 | 630 | 816 | 124 |
| citrate, pH 5.3, 150 mM | 40 | 563 | 1512 | 488 | 1308 | NT | NT |
| ArgHCl, 25 mM | | | | | | | |
| methionine, 0.05% PS-80 | | | | | | | |
| (1 mL in BD Hypak | | | | | | | |
| syringe, 27 G needle) | | | | | | | |
| Ref buffer vial | 5 | 47 | NT | 88 | 4 | NT | 0 |
| | 25 | 47 | NT | 48 | 11 | NT | 2 |
| | 40 | 47 | NT | 10 | 32 | NT | NT |
| Ref buffer PFS | 5 | 10 | NT | 10 | 6 | NT | 0 |
| | 25 | 10 | NT | 10 | 2 | NT | 6 |
| | 40 | 10 | NT | 34 | 4 | NT | NT |

However, these SVP are thought to primarily arise from the handling and processing of STX-100 drug substance (DS) during labscale UF/DF process, pre-fill storage, shipping to testing laboratory, and likely issues with the testing method. The growth rate of SVP>10 um (picked up more sensitively by MFI) do not suggest significant instability in any of the arginine-HCl containing formulations except 18169-67 that contains 200 mM trehalose. SVP counts are also observed to be higher in the formulations in pre-filled syringe presentation compared to vial presentation. This indicates that the testing method also identified a significant amount of silicone oil micro-droplets that commonly occur in such syringes. SVP analysis via HIAC method (as per USP-788) that is based on light-obscuration did not indicate instability at the desired storage condition of 5° C. (Tables 7 and 8).

TABLE 7
Sub-visible particulates (>10 um)/mL (HIAC)

| | | Particulates (>10 um); <=6000 | | | | | |
|---|---|---|---|---|---|---|---|
| | | Time (months) | | | | | |
| Lot number | Temperature | 0 | 1 | 3 | 6 | 9 | 12 |
| # 18169-62 | 5 | 23 | 32 | 22 | 46 | 61 | 63 |
| 150 mg/mL, 20 mM | 25 | 23 | 111 | 175 | 55 | 645 | 936 |
| citrate, pH 5.3, 150 mM | 40 | 23 | 1594 | 3574 | 83 | NT | NT |
| ArHCl, 0.05% PS-80 | | | | | | | |
| (1 mL in 3 mL Schott | | | | | | | |
| vial) | | | | | | | |
| # 18169-64 | 5 | 74 | 7 | 92 | 69 | 107 | 63 |
| 150 mg/mL, 20 mM | 25 | 74 | 185 | 246 | 464 | 732 | 548 |
| citrate, pH 5.3, 150 mM | 40 | 74 | 3910 | 5749 | 213 | NT | NT |
| ArHCl, 0.05% PS-80 | | | | | | | |
| (1 mL in BD Hypak | | | | | | | |
| syringe, 27 G needle) | | | | | | | |
| # 18169-66 | 5 | 57 | 56 | 104 | 77 | 138 | 40 |
| 150 mg/mL, 20 mM | 25 | 57 | 80 | 526 | 260 | 709 | 743 |
| citrate, pH 5.3, 150 mM | 40 | 57 | 4126 | 10947 | 267 | NT | NT |
| ArHCl, 25 mM | | | | | | | |
| methionine, 0.05% PS-80 | | | | | | | |
| (1 mL in BD Hypak | | | | | | | |
| syringe, 27 G needle) | | | | | | | |
| # 18169-67 | 5 | 71 | 83 | 92 | 53 | 215 | 137 |
| 150 mg/mL, 20 mM | 25 | 71 | 179 | 4292 | 289 | 387 | 1904 |
| citrate, pH 5.3, 200 mM | 40 | 71 | 1161 | 4993 | 184 | NT | NT |
| trehalose, 0.05% PS-80 | | | | | | | |
| (1 mL in BD Hypak | | | | | | | |
| syringe, 27 G needle) | | | | | | | |
| # 18169-72 | 5 | 51 | 72 | 141 | 96 | 280 | 37 |
| 250 mg/mL, 20 mM | 25 | 51 | 132 | 117 | 105 | 442 | 77 |
| citrate, pH 5.3, 150 mM | 40 | 51 | 72 | 105 | 232 | NT | NT |
| ArgHCl, 25 mM | | | | | | | |
| methionine, 0.05% PS-80 | | | | | | | |
| (1 mL in BD Hypak | | | | | | | |
| syringe, 27 G needle) | | | | | | | |
| Ref buffer vial | 5 | 41 | NT | 92 | 77 | NT | 48 |
| | 25 | 41 | NT | 101 | 53 | NT | 61 |
| | 40 | 41 | NT | 44 | 98 | NT | NT |
| Ref buffer PFS | 5 | 233 | NT | 251 | 325 | NT | 111 |
| | 25 | 233 | NT | 224 | 416 | NT | 231 |
| | 40 | 233 | NT | 415 | 714 | NT | NT |

TABLE 8
Sub-visible particulates (>25 um)/mL (HIAC)

| | | Particulates (>25 um): <=600 | | | | | |
|---|---|---|---|---|---|---|---|
| | | Time (months) | | | | | |
| Lot number | Temperature | 0 | 1 | 3 | 6 | 9 | 12 |
| # 18169-62 | 5 | 2 | 0 | 1 | 0 | 2 | 0 |
| 150 mg/mL, 20 mM | 25 | 2 | 1 | 1 | 0 | 7 | 5 |
| citrate, pH 5.3, 150 mM | 40 | 2 | 47 | 44 | 0 | NT | NT |
| ArHCl, 0.05% PS-80 | | | | | | | |
| (1 mL in 3 mL Schott | | | | | | | |
| vial) | | | | | | | |
| # 18169-64 | 5 | 1 | 0 | 1 | 0 | 2 | 0 |
| 150 mg/mL, 20 mM | 25 | 1 | 3 | 5 | 3 | 30 | 5 |
| citrate, pH 5.3, 150 mM | 40 | 1 | 101 | 118 | 8 | NT | NT |
| ArHCl, 0.05% PS-80 | | | | | | | |
| (1 mL in BD Hypak | | | | | | | |
| syringe, 27 G needle) | | | | | | | |
| # 18169-66 | 5 | 1 | 2 | 3 | 3 | 2 | 2 |
| 150 mg/mL, 20 mM | 25 | 1 | 6 | 10 | 6 | 30 | 3 |
| citrate, pH 5.3, 150 mM | 40 | 1 | 77 | 482 | 18 | NT | NT |
| ArHCl, 25 mM | | | | | | | |
| methionine, 0.05% PS-80 | | | | | | | |
| (1 mL in BD Hypak | | | | | | | |
| syringe, 27 G needle) | | | | | | | |
| # 18169-67 | 5 | 1 | 4 | 2 | 0 | 10 | 1 |
| 150 mg/mL, 20 mM | 25 | 1 | 5 | 102 | 10 | 10 | 16 |
| citrate, pH 5.3, 200 mM | 40 | 1 | 21 | 75 | 12 | NT | NT |
| trehalose, 0.05% PS-80 | | | | | | | |
| (1 mL in BD Hypak | | | | | | | |
| syringe, 27 G needle) | | | | | | | |
| # 18169-72 | 5 | 2 | 4 | 5 | 13 | 35 | 2 |
| 250 mg/mL, 20 mM | 25 | 2 | 11 | 8 | 2 | 32 | 7 |
| citrate, pH 5.3, 150 mM | 40 | 2 | 7 | 4 | 20 | NT | NT |
| ArgHCl, 25 mM | | | | | | | |
| methionine, 0.05% PS-80 | | | | | | | |
| (1 mL in BD Hypak | | | | | | | |
| syringe, 27 G needle) | | | | | | | |
| Ref buffer vial | 5 | 0 | NT | 1 | 0 | NT | 0 |
| | 25 | 0 | NT | 0 | 0 | NT | 0 |
| | 40 | 0 | NT | 0 | 1 | NT | NT |
| Ref buffer PFS | 5 | 2 | NT | 0 | 0 | NT | 0 |
| | 25 | 2 | NT | 3 | 7 | NT | 4 |
| | 40 | 2 | NT | 8 | 15 | NT | NT |

Oxidation data: Forced oxidation analysis in the past on STX-100 samples had revealed oxidation propensity in Met-55 contained in the second heavy chain CDR along with two other methionines (Met-255 and Met-431) in the Fc region. Structure-activity relationship studies revealed that oxidation in these residues do not lead to any change in binding activity to the antigen. In this study, it was also investigated whether oxidation in these residues over time leads to instability due to the presence of polysorbate-80 as a likely oxidizing agent. The % oxidation was determined using a LCMS method after generating in Met residues contained in corresponding peptides generated (Met-55 in peptide H2, Met-255 in peptide H15, and Met-431 in peptide H30) by LysC cleavage. Overall, there was no major increase in oxidation at each site although the presence of methionine as an excipient in the formulation did suppress this oxidation reaction (Tables 9A and 9B).

TABLE 9A
Oxidation analysis on formulations stored at 5° C.

| | t = 0 | | | t = 6 months at 5° C. | | | t = 12 months at 5° C. | | |
|---|---|---|---|---|---|---|---|---|---|
| Lot# | % H2—Ox | % H15—Ox | % H30—Ox | % H2—Ox | % H15—Ox | % H30—Ox | % H2—Ox | % H15—Ox | % H30—Ox |
| #18169-62 | 7.9 | 7.9 | 5.9 | 11.2 | 9.7 | 6.7 | 8.40 | 7.20 | 4.90 |
| 150 mg/mL, | | | | | | | | | |
| 20 mM citrate, | | | | | | | | | |
| pH 5.3, 150 | | | | | | | | | |
| mM ArHCl, | | | | | | | | | |
| 0.05% PS-80 | | | | | | | | | |
| (1 mL in 3 mL | | | | | | | | | |
| Schott vial) | | | | | | | | | |
| #18169-64 | 7.9 | 8.1 | 6.2 | 10.3 | 9.4 | 5.9 | 8.70 | 7.40 | 5.00 |
| 150 mg/mL, | | | | | | | | | |
| 20 mM citrate, | | | | | | | | | |
| pH 5.3, 150 | | | | | | | | | |
| mM ArHCl, | | | | | | | | | |
| 0.05% PS-80 | | | | | | | | | |
| (1 mL in BD | | | | | | | | | |
| Hypak syringe, | | | | | | | | | |
| 27 G needle) | | | | | | | | | |
| #18169-66 | 7.8 | 8.2 | 6.3 | 8.5 | 8.7 | 5.3 | 7.00 | 7.00 | 4.90 |
| 150 mg/mL, | | | | | | | | | |
| 20 mM citrate, | | | | | | | | | |
| pH 5.3, 150 | | | | | | | | | |
| mM ArHCl 25 | | | | | | | | | |
| mM methionine, | | | | | | | | | |
| 0.05% PS-80 | | | | | | | | | |
| (1 mL in BD | | | | | | | | | |
| Hypak syringe, | | | | | | | | | |
| 27 G needle) | | | | | | | | | |
| # 18169-67 | 8.1 | 8.3 | 6.5 | 9.6 | 9.5 | 5.8 | 7.50 | 7.10 | 4.90 |
| 150 mg/mL, | | | | | | | | | |
| 20 mM citrate, | | | | | | | | | |
| pH 5.3, 200 | | | | | | | | | |
| mM trehalose, | | | | | | | | | |
| 0.05% PS-80 | | | | | | | | | |
| (1 mL in BD | | | | | | | | | |
| Hypak syringe, | | | | | | | | | |
| 27 G needle) | | | | | | | | | |
| # 18169-72 | 7 | 5.7 | 3.4 | 9 | 7.8 | 4.2 | 6.60 | 5.10 | 2.60 |
| 250 mg/mL, | | | | | | | | | |
| 20 mM citrate, | | | | | | | | | |
| pH 5.3, 150 | | | | | | | | | |
| mM ArgHCl, 25 | | | | | | | | | |
| mM methionine, | | | | | | | | | |
| 0 05% PS-80 | | | | | | | | | |
| (1 mL in BD | | | | | | | | | |
| Hypak syringe. | | | | | | | | | |
| 27 G needle | | | | | | | | | |

TABLE 9B
Oxidation analysis on formulations stored at 25° C.

| | t = 0 | | | t = 6 months at 25° C. | | | t = 12 months at 25° C. | | |
|---|---|---|---|---|---|---|---|---|---|
| Lot# | % H2—Ox | % H15—Ox | % H30—Ox | % H2—Ox | % H15—Ox | % H30—Ox | % H2—Ox | % H15—Ox | % H30—Ox |
| #18169-62 | 7.9 | 7.9 | 5.9 | 17.7 | 10.4 | 6.8 | 18.40 | 8.50 | 5.80 |
| 150 mg/mL, | | | | | | | | | |
| 20 mM citrate, | | | | | | | | | |
| pH 5.3, 150 | | | | | | | | | |
| mM ArHCl, | | | | | | | | | |
| 0.05% PS-80 | | | | | | | | | |
| (1 mL in 3 mL | | | | | | | | | |
| Schott vial) | | | | | | | | | |
| #18169-64 | 7.9 | 8.1 | 6.2 | 17 | 10.2 | 6.2 | 18.40% | 8.40 | 5.60 |
| 150 mg/mL, | | | | | | | | | |
| 20 mM citrate, | | | | | | | | | |
| pH 5.3, 150 | | | | | | | | | |
| mM ArHCl, | | | | | | | | | |
| 0.05% PS-80 | | | | | | | | | |
| (1 mL in BD | | | | | | | | | |
| Hypak syringe, | | | | | | | | | |
| 27 G needle) | | | | | | | | | |
| #18169-66 | 7.8 | 8.2 | 6.3 | 10.2 | 8.8 | 5.8 | 9.00% | 7.20 | 5.20 |
| 150 mg/mL, | | | | | | | | | |
| 20 mM citrate, | | | | | | | | | |
| pH 5.3, 150 | | | | | | | | | |
| mM ArHCl 25 | | | | | | | | | |
| mM methionine, | | | | | | | | | |
| 0.05% PS-80 | | | | | | | | | |
| (1 mL in BD | | | | | | | | | |
| Hypak syringe, | | | | | | | | | |
| 27 G needle) | | | | | | | | | |
| # 18169-67 | 8.1 | 8.3 | 6.5 | 13.6 | 9.8 | 6.2 | 12.90% | 8.10 | 5.50 |
| 150 mg/mL, | | | | | | | | | |
| 20 mM citrate, | | | | | | | | | |
| pH 5.3, 200 | | | | | | | | | |
| mM trehalose, | | | | | | | | | |
| 0.05% PS-80 | | | | | | | | | |
| (1 mL in BD | | | | | | | | | |
| Hypak syringe, | | | | | | | | | |
| 27 G needle) | | | | | | | | | |
| # 18169-72 | 7 | 5.7 | 3.4 | 10.8 | 7.8 | 4 | 9.10% | 5.70 | 2.90 |
| 250 mg/mL, | | | | | | | | | |
| 20 mM citrate, | | | | | | | | | |
| pH 5.3, 150 | | | | | | | | | |
| mM ArgHCl, 25 | | | | | | | | | |
| mM methionine, | | | | | | | | | |
| 0 05% PS-80 | | | | | | | | | |
| (1 mL in BD | | | | | | | | | |
| Hypak syringe. | | | | | | | | | |
| 27 G needle | | | | | | | | | |

Visible particulate data: Appearance (particulate) observations did not reveal any significant increase in visible particulates for any formulation throughout the 12-month storage period at 5° C. (Table 10A). Visible particulates do appear at 25° C. over long-term storage (Table 10B) and are probably linked to the increase in large SVP (>25 um) at this temperature.

TABLE 10A
Long-term assessment of visible particulates at 5° C.

| | Months at 5° C. | | | | | | |
|---|---|---|---|---|---|---|---|
| Lot# | 0 | 1 | 2 | 3 | 6 | 9 | 12 |
| #18169-62 | No visible | No visible | No visible | No visible | No visible | No visible | *No visible |
| 150 mg/mL, | particulates | particulates | particulates | particulates | particulates | particulates | particulates |
| 20 mM citrate, | observed | observed | observed | observed | observed | observed | observed |
| pH 5.3, 150 | | | | | | | |
| mM ArHCl, | | | | | | | |
| 0.05% PS-80 | | | | | | | |
| (1 mL in 3 mL | | | | | | | |
| Schott vial) | | | | | | | |
| #18169-64 | No visible | No visible | No visible | No visible | No visible | No visible | No visible |
| 150 mg/mL, | particles | particulates | particulates | particulates | particulates | particulates | particulates |
| 20 mM citrate, | observed | observed | observed | observed | observed | observed | observed |
| pH 5.3, 150 | | | | | | | |
| mM ArHCl, | | | | | | | |
| 0.05% PS-80 | | | | | | | |
| (1 mL in BD | | | | | | | |
| Hypak syringe, | | | | | | | |
| 27 G needle) | | | | | | | |
| #18169-66 | No visible | No visible | No visible | No visible | No visible | No visible | *No visible |
| 150 mg/mL, | particles | particulates | particulates | particulates | particulates | particulates | particulates |
| 20 mM citrate, | observed | observed | observed | observed | observed | observed | observed |
| pH 5.3, 150 | | | | | | | |
| mM ArHCl 25 | | | | | | | |
| mM methionine, | | | | | | | |
| 0.05% PS-80 | | | | | | | |
| (1 mL in BD | | | | | | | |
| Hypak syringe, | | | | | | | |
| 27 G needle) | | | | | | | |
| # 18169-67 | No visible | No visible | No visible | No visible | No visible | No visible | No visible |
| 150 mg/mL, | particles | particulates | particulates | particulates | particulates | particulates | particulates |
| 20 mM citrate, | observed | observed | observed | observed | observed | observed | observed |
| pH 5.3, 200 | | | | | | | |
| mM trehalose, | | | | | | | |
| 0.05% PS-80 | | | | | | | |
| (1 mL in BD | | | | | | | |
| Hypak syringe, | | | | | | | |
| 27 G needle) | | | | | | | |
| # 18169-72 | No visible | No visible | No visible | No visible | No visible | No visible | *No visible |
| 250 mg/mL, | particles | particles | particles | particles | particulates | particulates | particulates |
| 20 mM citrate, | observed | observed | observed | observed | observed | observed | observed |
| pH 5.3, 150 | | | | | | | |
| mM ArgHCl, 25 | | | | | | | |
| mM methionine, | | | | | | | |
| 0 05% PS-80 | | | | | | | |
| (1 mL in BD | | | | | | | |
| Hypak syringe. | | | | | | | |
| 27 G needle | | | | | | | |
| Ref Buffer vial | No visible | NT | NT | No visible | White flake | NT | No visible |
| | particles | | | particles | particle | | particles |
| | observed | | | observed | | | observed |
| Ref Buffer PFS | No visible | NT | NT | No visible | No visible | NT | No visible |
| | particles | | | particles | particles | | particles |
| | observed | | | observed | observed | | observed |

*Samples were re-examined after initial report revealed some particulates. The internal examinations on triplicate vials/syringes failed to show any visible particulates. The initial observations are therefore thought to arise from error in handling or human error.

TABLE 10B
Long-term assessment of visible particulates at 25° C.

| | Months at 25° C. | | | | | | |
|---|---|---|---|---|---|---|---|
| Lot# | 0 | 1 | 2 | 3 | 6 | 9 | 12 |
| #18169-62 | No visible | No visible | No visible | No visible | No visible | No visible | No visible |
| 150 mg/mL, | particulates | particulates | particulates | particulates | particulates | particulates | particulates |
| 20 mM citrate, | observed | observed | observed | observed | observed | observed | observed |
| pH 5.3, 150 | | | | | | | |
| mM ArHCl, | | | | | | | |
| 0.05% PS-80 | | | | | | | |
| (1 mL in 3 mL | | | | | | | |
| Schott vial) | | | | | | | |
| #18169-64 | No visible | No visible | No visible | No visible | No visible | No visible | No visible |
| 150 mg/mL, | particles | particulates | particulates | particulates | particulates | particulates | particulates |
| 20 mM citrate, | observed | observed | observed | observed | observed | observed | observed |
| pH 5.3, 150 | | | | | | | |
| mM ArHCl, | | | | | | | |
| 0.05% PS-80 | | | | | | | |
| (1 mL in BD | | | | | | | |
| Hypak syringe, | | | | | | | |
| 27 G needle) | | | | | | | |
| #18169-66 | No visible | No visible | No visible | No visible | Two other | White fiber | Small rounc |
| 150 mg/mL, | particles | particulates | particulates | particulates | white | like | particulate |
| 20 mM citrate, | observed | observed | observed | observed | particles | particulates | |
| pH 5.3, 150 | | | | | | observed | |
| mM ArHCl 25 | | | | | | | |
| mM methionine, | | | | | | | |
| 0.05% PS-80 | | | | | | | |
| (1 mL in BD | | | | | | | |
| Hypak syringe, | | | | | | | |
| 27 G needle) | | | | | | | |
| # 18169-67 | No visible | No visible | No visible | No visible | No visible | No visible | Small fiber |
| 150 mg/mL, | particles | particulates | particulates | particulates | particulates | particulates | like |
| 20 mM citrate, | observed | observed | observed | observed | observed | observed | particulate |
| pH 5.3, 200 | | | | | | | |
| mM trehalose, | | | | | | | |
| 0.05% PS-80 | | | | | | | |
| (1 mL in BD | | | | | | | |
| Hypak syringe, | | | | | | | |
| 27 G needle) | | | | | | | |
| # 18169-72 | No visible | No visible | No visible | No visible | No visible | No visible | No visible |
| 250 mg/mL, | particles | particles | particles | particles | particulates | particulates | particulates |
| 20 mM citrate, | observed | observed | observed | observed | observed | observed | observed |
| pH 5.3, 150 | | | | | | | |
| mM ArgHCl, 25 | | | | | | | |
| mM methionine, | | | | | | | |
| 0 05% PS-80 | | | | | | | |
| (1 mL in BD | | | | | | | |
| Hypak syringe. | | | | | | | |
| 27 G needle | | | | | | | |
| Ref Buffer vial | No visible | NT | NT | White fiber | No visible | NT | No visible |
| | particles | | | and 10 other | particulates | | particulates |
| | observed | | | particles | observed | | observed |
| Ref Buffer PFS | No visible | NT | NT | No visible | No visible | NT | No visible |
| | particles | | | particles | particles | | particles |
| | observed | | | observed | observed | | observed |

Milestone assays: Other stability assays such as CE-SDS (non-reduced), icIEF, osmolality, viscosity and potency tested at t=0, 6, 12 months are grouped herein as milestone assays. This data does not reveal any significant degradation in the samples at 5° C. over 12 months (Tables 11A through 11E).

TABLE 11A
Milestone assays performed at t = 0

| | | Formulation | | | | |
|---|---|---|---|---|---|---|
| Assay (t = 0) | | 18169-62 | 18169-64 | 18169-66 | 18169-67 | 18169-72 |
| CE-SDS | % Purity | 95.68 | 95.315 | 95.58 | 95.435 | 93.735 |
| (non- | % Single Largest | 1.75 | 1.825 | 1.78 | 1.8 | 3.245 |
| reduced) | Impurity | | | | | |
| iCIEF | % Main Peak | 53 | 53 | 53.55 | 52.85 | 55.2 |
| | % Acidic | 41.75 | 42.3 | 41.55 | 42.35 | 40.15 |
| | Isoform | | | | | |
| | % Basic Isoform | 5.2 | 4.75 | 4.9 | 4.75 | 4.65 |
| Osmolality | mOsm/kg | 368 | 4330 | 417 | 350 | 417 |
| (Freezing pt) | | | | | | |
| Viscosity (cP) at 25° C. | | 6.14 | 6.49 | 6.73 | 9.64 | 46.7 |
| Potency | % Relative | 103 | 106 | 95 | 96 | 96 |
| | potency | (109, 97 | (120, 95) | (102, 88) | (102, 91) | (107, 86) |
| | (95% UCL, | | | | | |
| | 95% LCL) | | | | | |

TABLE 11B
Milestone assays performed at t = 6 months on formulations stored at 5° C.

| | | Formulation | | | | |
|---|---|---|---|---|---|---|
| Assay (t = 6 M) 5° C. | | 18169-62 | 18169-64 | 18169-66 | 18169-67 | 18169-72 |
| CE-SDS | % Purity | 94.49 | 94.76 | 94.82 | 94.62 | 92.9 |
| (non- | % Single Largest | 1.86 | 1.83 | 1.8 | 1.77 | 3.08 |
| reduced) | Impurity | | | | | |
| iCIEF | % Main Peak | 51.6 | 52.1 | 52.5 | 49.6 | 54.3 |
| | % Acidic | 42.5 | 41.7 | 41.6 | 44.2 | 40.5 |
| | Isoform | | | | | |
| | % Basic Isoform | 5.9 | 6.2 | 5.9 | 6.1 | 5.3 |
| Osmolality | mOsm/kg | NT | NT | NT | NT | NT |
| (Freezing pt) | | | | | | |
| Viscosity (cP) at 5° C. | | 14 | 13.9 | 14.8 | 24 | 146.5 |
| Potency | % | 98 | 105 | 96 | 114 | 107 |
| | | (88, 108) | (89, 125) | (77, 120) | (108, 119) | (92, 123) |

TABLE 11C
Milestone assays performed at t = 6 months on formulations stored at 25° C.

| | | Formulation | | | | |
|---|---|---|---|---|---|---|
| Assay (t = 6 M) 25° C. | | 18169-62 | 18169-64 | 18169-66 | 18169-67 | 18169-72 |
| CE-SDS | % Purity | 90.5 | 90.4 | 90.43 | 91.55 | 90.04 |
| (non- | % Single Largest | 2.13 | 2.11 | 2.09 | 2.02 | 3.48 |
| reduced) | Impurity | | | | | |
| iCIEF | % Main Peak | 41.6 | 41.8 | 42.2 | 39.6 | 43.9 |
| | % Acidic | 48.6 | 48.3 | 48 | 51.8 | 46.2 |
| | Isoform | | | | | |
| | % Basic Isoform | 9.8 | 9.9 | 9.8 | 8.6 | 9.9 |
| Osmolality | mOsm/kg | NT | NT | NT | NT | NT |
| (Freezing pt) | | | | | | |
| Viscosity (cP) at 25° C. | | 6.3 | 6.4 | 6.4 | 9.3 | 46.2 |
| Potency | % | 87 | 89 | 92 | 93 | 95 |
| | | (85, 89) | (85, 92) | (91, 94) | (86, 102) | (93, 97) |

TABLE 11D
Milestone assays performed at t = 12 months on formulations stored at 5° C.

| | | Formulation | | | | |
|---|---|---|---|---|---|---|
| Assay (t = 12 M) 5° C. | | 18169-62 | 18169-64 | 18169-66 | 18169-67 | 18169-72 |
| CE-SDS | % Purity | 95.21 | 95.03 | 94.95 | 94.69 | 92.88 |
| (non- | % Single Largest | 1.61 | 1.7 | 1.75 | 1.88 | 3.44 |
| reduced) | Impurity | | | | | |
| iCIEF | % Main Peak | 51.6 | 51.5 | 51 | 51.1 | 53.4 |
| | % Acidic | 42.5 | 42.5 | 42.9 | 43.2 | 41.2 |
| | Isoform | | | | | |
| | % Basic Isoform | 5.9 | 6 | 6.1 | 5.7 | 5.4 |
| Osmolality | mOsm/kg | 360 | 170 | 181 | 146 | 182 |
| (Freezing pt) | | | | | | |

| | | | | | |
|---|---|---|---|---|---|
| Viscosity (cP) at 5° C. | 15.2 | 14.4 | 15 | 24.5 | 152.6 |

TABLE 11E
Milestone assays performed at t = 12 months on formulations stored at 25° C.

| | | Formulation | | | | |
|---|---|---|---|---|---|---|
| Assay (t = 12 M) 25° C. | | 18169-62 | 18169-64 | 18169-66 | 18169-67 | 18169-72 |
| CE-SDS | % Purity | 87.32 | 87.25 | 87.24 | 88.72 | 87.35 |
| (non- | % Single Largest | 2.85 | 2.94 | 2.92 | 2.46 | 4.24 |
| reduced) | Impurity | | | | | |
| iCIEF | % Main Peak | 33.5 | 34 | 34.1 | 30.3 | 34.9 |
| | % Acidic | 55.3 | 54.9 | 54.8 | 60.6 | 53.3 |
| | Isoform | | | | | |
| | % Basic Isoform | 11.3 | 11 | 11.1 | 9.1 | 11.8 |
| Osmolality | mOsm/kg | 370 | 169 | 182 | 130 | 174 |
| (Freezing pt) | | | | | | |

| | | | | | |
|---|---|---|---|---|---|
| Viscosity (cP) at 25° C. | 15.2 | 6.5 | 6.71 | 6.69 | 9.79 |

Color, Clarity and pH Data: The visually observed color in all formulations except 18169-72 remained below BY3 (BY4-BY5 or BY3-BY4) throughout the period of 1 year at 5° C. The color of 18169-72 was between BY3-BY4 up to 9 months and was observed to be BY3-BY2 at 12 months. The clarity of all formulations remained below 30 NTU (6-18 NTU or 18-30 NTU) throughout the storage at 5° C.
Conclusion: Formulations 18169-62, 18169-64, 18169-66 and 18169-72 were found to be stable within acceptable limits over 1 year. The trends in the most critical attributes: % total aggregates and Sub-visible particulates (HIAC) over this time period suggest that formulation 18169-64 and 18169-66 are both suitable to be pursued for a pre-filled syringe (PFS) drug product (DP).

### Example 3: Characterization of the Viscosity of the Formulation

The impact of pH and methionine concentration on the viscosity of STX-100 formulation at high concentration was evaluated using a full-factorial design of experiment (DOE) study. The following formulation parameters were varied:
1) pH: 5.0, 5.5, 6.0
2) Methionine: 0, 10, 25 mM
3) Protein concentration: 150, 220, 240 and 260 mg/mL
   The core formulation buffer was: 20 mM Citric acid/Na-citrate, 150 mM arginine HCl, 0.05% PS80.

The data indicated that the viscosity of STX-100 formulation was not significantly impacted by either the pH or the methionine concentration around a core formulation containing 20 mM Na-citrate/citric acid, 150 mM Arginine-HCl, 0.05% PS-80. Both the 5′C and 25° C. data did not reveal p-values lower than 0.05 for each of the two formulation parameters. The only solution parameter with a significant impact on viscosity was the protein concentration which was expected in the range examined. These results show that the pH and excipient levels can be varied within this design space without negatively impacting the viscosity of the formulation.

### Example 4: Drug Product Bracketing Study

This study was done to examine the effect of increasing the methionine content from 5 mM to 10 mM as well as lowering the polysorbate-80 level from 0.05% to 0.03% on long-term stability attributes.

The following two formulations were prepared and filled into representative pre-filled syringes (0.8 mL fill in BD Hypak STW 27G PFS).

Formulation A: 150 mg/mL STX-100, 20 mM Na-citrate/citric acid, pH 5.5, 150 mM Arginine-HCl, 10 mM Methionine, 0.05% polysorbate-80.

Formulation B: 150 mg/mL STX-100, 20 mM Na-citrate/citric acid, pH 5.5, 150 mM Arginine-HCl, 5 mM Methionine, 0.03% polysorbate-80.

The results from long-term stability at 2-8° C. displayed equivalent stability based on the trends in % HMW and sub-visible particulates. Stability data was also collected at 25° C. and 40° C. for information purposes. The formulations did not appear significantly different in their oxidized species content.

Thus, the data shows flexibility in polysorbate-80 and methionine concentration for the formulation.

### Example 5: Process Stability Study

This study assessed the impact of different polysorbate-80 surfactant levels on stability of STX-100 in small-scale DS containers (PC bottles or bags), and representative DP in pre-filled syringes (PFS). The formulation was subjected to two different stresses:

a) Multiple freeze-thaw cycles (1, 3 and 5 freeze-thaw cycles),

b) Shaking-induced agitation stress (orbital shaking at 650 rpm for 72 hours at ambient conditions) and

c) Representative ambient hold-times (selected PS-80 level only).

The different PS-80 levels selected for evaluations were 0, 0.01, 0.02, 0.05, 0.08, 0.1% w/v in 150 mg/mL STX-100 formulation containing 20 mM Na-citrate/citric acid, pH 5.5, 150 mM Arginine-HCl, 5 mM Methionine. The container closure system used for the evaluations were Polycarbonate bottles (1 mL fill in 5 mL bottle), Small DS bag (30 mL capacity, 5 or 15 mL fill), PFS syringes (BD Hypak 47368319 with plungers (47165919) filled with either 0.8 mL or 0.3 mL at 150 mg/mL or 0.3 mL at 40 mg/mL).

The product quality attributes examined were: Visible appearance (particulates), Turbidity (A340), % Total aggregates (SEC), Protein concentration (SoloVPE method), and Sub-visible particulates (MFI)

The results from target drug product fill volume of 0.8 mL at 150 mg/mL STX-100 showed that agitating the STX-100 syringes at 650 rpm for 72 hours at ambient temperature protected from light has minimal impact on the visible particulates as long as there is 0.01% PS-80 present in the formulation. One dust-like particle was observed in the 0.02% PS80 sample but this appears to be environmental. There was only a 0.05-0.1% increase in soluble aggregate after agitation in the formulations containing 0-0.01% PS80 while no observable increase in soluble aggregate in any other formulations. The turbidity data indicated no substantial increase in OD340 for all formulations except the one with 0.1% PS80 indicating some contribution from a relatively high level of PS80. However, the SVP data indicates no substantial particle formation tendency as long as PS80 is present. The process study results suggested that 0.05% w/v was an optimal level of polysorbate-80 to protect the formulation against freeze-thaw stress, agitation stress, and process hold times. A suggested specification for PS80 level for product development purposes is 0.05+/−0.025% w/v.

### Example 6: Selection of Formulation

Based on all the above studies the following STX-100 formulation displayed acceptable stability over long-term storage (1 year at 2-8° C.), worst-case agitation stress (650 rpm for 72 h) and worst-case freeze-thaw stress (5 freeze-thaw cycles): 150 mg/mL STX-100, 20 mM Na-citrate/citric acid, 150 mM Arginine-HCl, 5 mM Methionine, 0.05% w/v polysorbate-80, pH 5.5.

Based on trends in stability attributes, this formulation guarantees greater than 24 month stability at 2-8° C. in a representative pre-filled syringe product.

### Example 7: Stability of STX-100 Formulations Comprising Thiol Group Containing Excipients

The addition of thiol group containing excipients to an STX-100 formulation reduces aggregation as determined by the development of high molecular weight species during storage.

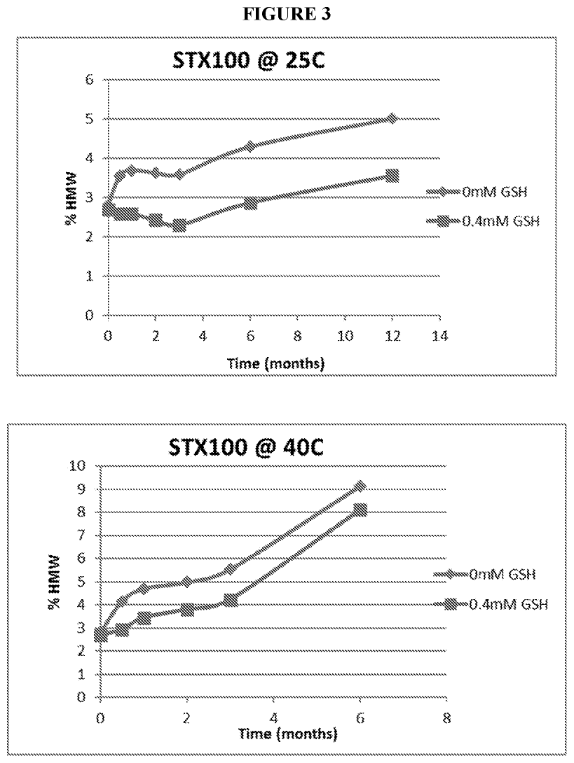

The control STX-100 formulation had 150 mg/mL STX-100, 20 mM citrate/citric acid, 150 mM L-Arginine HCl, 5 mM Methionine, 0.05% Polysorbate-80, pH 5.5. The control formulation was spiked with a thiol group containing excipient: GSH. The formulations were stored at 25° C. and 40° C. As shown in FIG. 3, the addition of GSH reduced aggregation during storage.

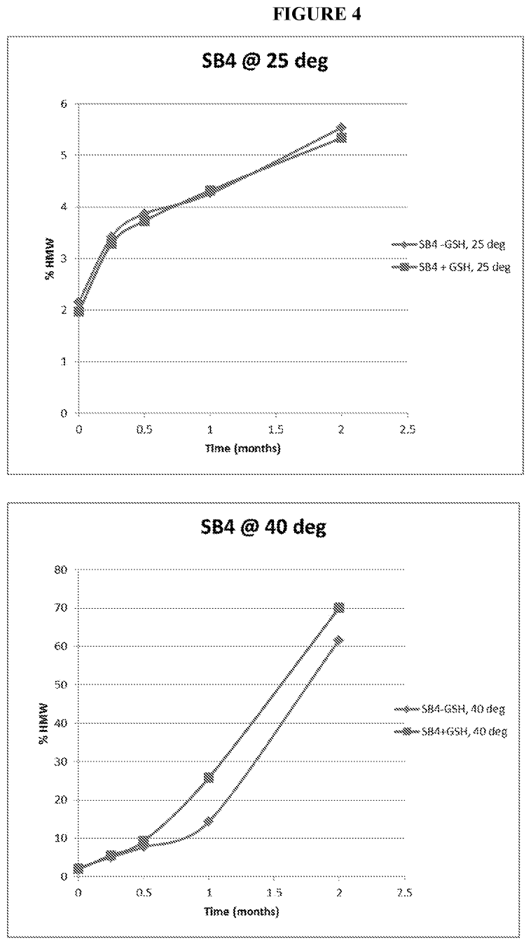

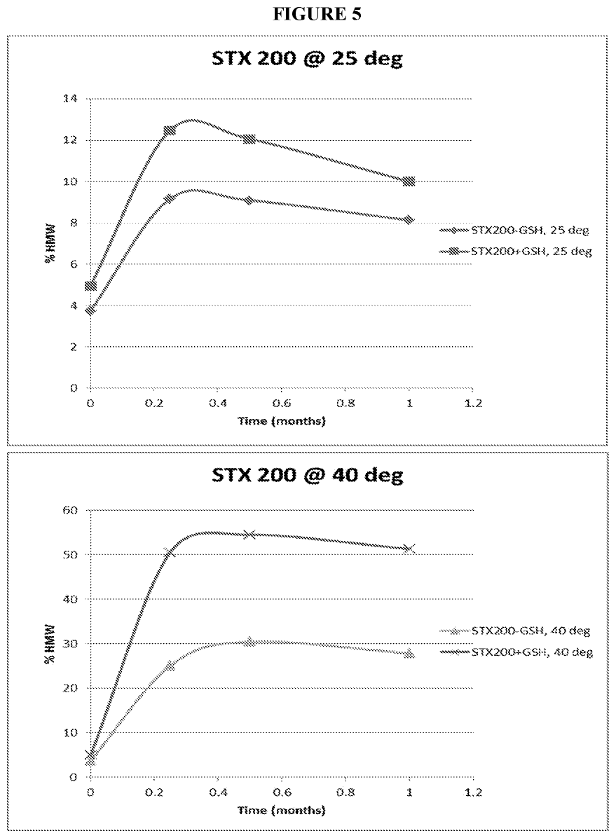

Addition of glutathione negatively impacted another antibody, STX200, where an increase in aggregation was observed (FIG. 5). STX 200 is an aglycosylated molecule, demonstrating poor conformational stability at higher temperatures. Hence, unfolding of the molecule exposes the thiol group making it more susceptible to crosslinking with the thiol in glutathione and promoting further aggregation. Glutathione did not have any effect on the aggregation kinetics of SB4 (BENEPALI®, an etanercept biosimilar referencing Enbrel®) at 25° C., but facilitated faster aggregation at 40° C. (FIG. 4).

### Example 8: Stability Data for STX-100 Formulations

Stability study data for 50 and 100 mg/mL STX-100 formulations in 20 mM sodium citrate buffer containing 150 mM Arg.HCL, 5 mM methionine, 0.05% PS80, at pH 5.5 filled into syringes (0.8 mL/syringe) supports stability for 36 months when stored at 2-8° C. This is based on stability data at the long term storage condition of 2-8° C. See Tables 12 and 13 below. Based upon this drug product data, a stability for 36 months can be assigned to a formulation at 70 mg/mL (0.8 mL/syringe) selected to deliver a dose of 56 mg.

TABLE 12
Stability Data for STX-100 Drug Product at 100 mg/mL in 1 mL Syringe, Stored at 2-8° C.

| Test/Attribute | Acceptance Criteria | 0 mo | 01 mo | 03 mo² | 06 mo² | 09 mo² | 12 mo² | 18 mo |
|---|---|---|---|---|---|---|---|---|
| Appearance - Clarity (NTU) | Report Results | 6 NTU < | 18 NTU < | N/A | N/A | N/A | N/A | 6 NTU < |
| | | Sample < | Sample < | | | | | Sample < |
| | | 18 NTU | 30 NTU | | | | | 18 NTU |
| Appearance - Clarity: LT 50 | Conforms | Conforms | Conforms | N/A | N/A | N/A | N/A | Conforms |
| NTU | | | | | | | | |
| Appearance - Color (BY Scale) | Report Results | 4 <= | 5 <= | N/A | N/A | N/A | N/A | 5 <= |
| | | Sample < 3 | Sample < 4 | | | | | Sample < 4 |
| Appearance - Color (BY Scale): | Conforms | Conforms | Conforms | N/A | N/A | N/A | N/A | Conforms |
| LT BY2 | | | | | | | | |
| Appearance - Essentially free of | Conforms | Conforms | Conforms | N/A | N/A | N/A | N/A | Conforms |
| visible particles | | | | | | | | |
| pH | 5.0-6.0 | 5.6 | 5.5 | N/A | N/A | N/A | N/A | 5.4 |
| Protein Concentration (RI) | 90-110 mg/ml | 100 | 100 | N/A | N/A | N/A | N/A | 99 |
| DELFIA Blocking - Binding | 75-133% | 101 | 100 | N/A | N/A | N/A | N/A | 96 |
| relative to Reference Standard | | | | | | | | |
| icIEF - Lower pl Isoforms (%) | Report Results | 43.3 | 39.8 | N/A | N/A | N/A | N/A | 43.9 |
| icIEF - Main pl Isoform (%) | Report Results | 53.3 | 55.1 | N/A | N/A | N/A | N/A | 48.3 |
| Size Exclusion | NMT 5.0% | 1.3 | 1.4 | N/A | N/A | N/A | N/A | 2.0 |
| Chromatography (SEC) - | | | | | | | | |
| Aggregates | | | | | | | | |
| Non-Reducing CE-SDS - | Report Results | 1.4 | 1.7 | N/A | N/A | N/A | N/A | 1.8 |
| Highest Single Impurity (%) | | | | | | | | |
| Non-Reducing CE-SDS - Total | NLT 90.0% | 96.5 | 95.2 | N/A | N/A | N/A | N/A | 95.6 |
| Purity | | | | | | | | |
| Endotoxin (USP, EP) - | NMT 130.00 EU/ml | <8.00 | N/S | N/S | N/A | N/A | N/A | <8.00 |
| Endotoxin | | | | | | | | |
| Particulates - NLT 10 um | NMT 6000 Counts/container | 165 | N/S | N/S | N/A | N/A | N/A | 76.59 |
| Particulates - NLT 25 um | NMT 600 Counts/container | 4 | N/S | N/S | N/A | N/A | N/A | 1.07 |
| Container Closure Integrity - | Conforms | Conforms¹ | N/S | N/S | N/A | N/A | N/A | Conforms |
| Seal Integrity | | | | | | | | |

TABLE 13
Stability Data for STX-100 Drug Product at 50 mg/mL in 1 mL Syringe, Stored at 2-8° C.

| Description: | PS TAB-14-10-033 (Cycle 2 drug product} stability data | | | | |
|---|---|---|---|---|---|
| Lot No.: | TR-PPD-009928 | Stability Protocol: | PSTAB-14-10-033 | Study Start Date: | 22 Oct. 1914 |
| Manufacturing Date | 16 Oct. 1914 | my CIMS Portocol: | TR-PPD-010008 | Concentration: | 50 mg/mL |
| Manufacturing Site | PPD, Cambridge | Storage Conditions: | 2-8° C. | Sample Container | PES 1 mL (0.8 mL fill) |

| | | Time Point (Months and TD Labware LIMS submission # | | | | | | | |
|---|---|---|---|---|---|---|---|---|---|
| | | | | | | | | PPD- | PPD- |
| | | 38228 | 38229 | 38230 | 38231 | 38232 | 38233 | 15-6943 | 15-6944 |
| Test Method | Acceptance Criteria¹ | 0 | 1 | 3 | 6 | 9 | 12 | 18 | 24 |
| Appearance | Color: Report Relults | BY6-BY5 | BY6-BY5 | BY6-BY5 | BY6-BY5 | BY5-BY4 | BY5-BY4 | N/A | BY7-BY6 |
| | Clarity. Report Results | 18-30 | 18-30 | 18-30 | 18-30 | 18-30 | 18-30 | N/A | 6-18 |
| | Essentialy free of visible | N/O | N/O | N/O | N/O | N/O | N/O | N/A | N/O |
| | particles | | | | | | | | |
| pH | 5.0-6.0 | 5.5 | 5.5 | 5.5 | 5.8 | 5.5 | 5.5 | N/A | 5.5 |
| Osmolality | Report Results | 332 | NT | NT | NT | NT | NT | NT | NT |
| Protein Conc. | 45-55 mg/mL | 52 | 52 | 51 | 52 | 52 | 51 | N/A | 52 |
| SEC | ≤5.0% Aggregates | 1.0 | 1.1 | 1. | 1.3 | 1.4 | 1.4 | N/A | 1.7 |
| ieIEF | Report % Lower pI | 43.5 | 42.8 | 42.3 | 42.4 | 43.8 | 41.0 | N/A | 43.0 |
| | Isoforms (xx.x %) | | | | | | | | |
| | Report % Main Peak | 49.8 | 50.5 | 51.2 | 49.6 | 48.8 | 53.1 | N/A | 50.4 |
| | (xx.x %) | | | | | | | | |
| | Report % Higher pI | 6.8 | 6.7 | 6.5 | 8.0 | 7.4 | 5.9 | N/A | 6.7 |
| | Informs (xx.x %) | | | | | | | | |
| Non-Redeucing | ≥90.0 % purity reuslts | 97.1 | 97.1 | 96.5 | 96.6 | 96.5 | 96.4 | N/A | 96.3 |
| CE-SDS | Report Highest Single | 1.7 | 1.7 | 1.7 | 1.7 | 1.6 | 1.8 | N/A | 1.7 |
| | Impurity (x.x %) | | | | | | | | |
| Reducing | Report % purity results | 97.1 | 97.3 | 96.5 | 96.7 | 96.8 | 96.0 | N/A | 96.6 |
| CE-SDS | Report Highest Single | 1.4 | 1.3 | 1.3 | 1.4 | 1.4 | 1.4 | N/A | 1.5 |
| | Impurity (x.x %) | | | | | | | | |
| Potency | 75 in [symbol] % Binding relative | [symbol] | N/A² | 102 | 98 | 95 | 100 | N/A | 106 |
| | to R [symbol] Standard | | | | | | | | |
| PS80 | Report % | 0.05 | NT | NT | NT | NT | 0.057 | NT | 0.05 |
| Oxidation | % H2—Ox | 5.3 | NT | 5.8 | 6.8 | NT | N/A² | N/A | 7 |
| | % H15—Ox | 4.9 | NT | 5.6 | 7.0 | NT | N/A² | N/A | 6.6 |
| | % H30—Ox | 2.9 | NT | 3.6 | 4.1 | NT | N/A² | N/A | 2.5 |
| Subvisible | ≥10 μm Particles: Report | 77 | 98 | 122 | 41 | 39 | 47 | N/A | 42 |
| Particles | Results, Particles/mL | | | | | | | | |
| (HIAC) | ≥25 μm Particles: Report | 37 | 0 | 5 | 0 | 0 | 0 | N/A | 0 |
| | Results, Particles/mL | | | | | | | | |
| Subvisible | ≥10 μm Particles: Report | 197 | NT | 21 | 400 | 150 | 235 | N/A | 43 |
| Particles | Results, Particles/mL³ | | | | | | | | |
| (MFI) | ≥25 μm Particles: Report | 13 | NT | 7 | 59 | 11 | 14 | N/A | 1 |
| | Results, Particles/mL³ | | | | | | | | |
| Subvisible | ≥10 μm Particles ≤ 6000 | 62 | 78 | 98 | 33 | 24 | 38 | N/A | 34 |
| Particles | Particles/Container | | | | | | | | |
| (HIAC)⁴ | ≥25 μm Particles ≤ 6000 | 30 | 0 | 4 | 0 | 0 | 0 | N/A | 0 |
| | Particles/Container | | | | | | | | |
| Subvisible | ≥10 μm Particles ≤ 6000 | 158 | NT | 17 | 320 | 120 | 188 | N/A | 34 |
| Particles | Particles/Container | | | | | | | | |
| (MFI)⁴ | ≥25 μm Particles ≤ 6000 | 10 | NT | 8 | 47 | 9 | 11 | N/A | 1 |
| | Particles/Container | | | | | | | | |

¹Based on platform specification and esperimental data. This Acceptance Criteria has not been approved and may be used For Information Only.
²Data is not available due to sample mishandling.
³T0 (0 month) data is from ELE: EXP-8 January 2015-0065;
⁴Calculated by using particles/mL × fill volume (0.8 mL)
N/O: No particulates observed;
NT: Not tested according to protocol;
N/A: Study was not performed due to program changes [symbol]
indicates data missing or illegible when filed

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A pharmaceutical composition comprising an anti-αvβ6 antibody or αvβ6-binding fragment thereof, and arginine hydrochloride (Arg.HCl), wherein the anti-αvβ6 antibody or αvβ6-binding fragment thereof comprises an immunoglobulin heavy chain variable domain (VH) and an immunoglobulin light chain variable domain (VL), the VH and VL, respectively, comprising:
(a) VH complementarity determining regions (CDRs), wherein
VH-CDR1 consists of the amino acid sequence set forth in SEQ ID NO:1;
VH-CDR2 consists of the amino acid sequence set forth in SEQ ID NO:2; and
VH-CDR3 consists of the amino acid sequence set forth in SEQ ID NO:3; and
(b) VL CDRs, wherein
VL-CDR1 consists of the amino acid sequence set forth in SEQ ID NO:4;
VL-CDR2 consists of the amino acid sequence set forth in SEQ ID NO:5; and
VL-CDR3 consists of the amino acid sequence set forth in SEQ ID NO:6, and
wherein the composition has a pH of 5.2 to 5.7.

2. The pharmaceutical composition of claim 1, wherein the composition comprises the anti-αvβ6 antibody or αvβ6-binding fragment thereof at a concentration of 50 mg/ml to 200 mg/ml.

3. The pharmaceutical composition of claim 1, wherein the composition comprises the anti-αvβ6 antibody or αvβ6-binding fragment thereof at a concentration of 100 mg/ml to 175 mg/ml.

4. The pharmaceutical composition of claim 1, wherein the composition comprises the anti-αvβ6 antibody or αvβ6-binding fragment thereof at a concentration of 150 mg/ml.

5. The pharmaceutical composition of any one of claims 1 to 4, wherein the composition comprises Arg.HCl at a concentration of 50 mM to 250 mM.

6. The pharmaceutical composition of any one of claims 1 to 4, wherein the composition comprises Arg.HCl at a concentration of 100 mM to 200 mM.

7. The pharmaceutical composition of any one of claims 1 to 4, wherein the composition comprises Arg.HCl at a concentration of 150 mM.

8. The pharmaceutical composition of any one of claims 1 to 7, wherein the composition comprises methionine.

9. The pharmaceutical composition of claim 8, wherein the composition comprises methionine at a concentration of 0.5 mM to 30 mM.

10. The pharmaceutical composition of claim 8, wherein the composition comprises methionine at a concentration of 1 mM to 10 mM.

11. The pharmaceutical composition of claim 8, wherein the composition comprises methionine at a concentration of 5 mM.

12. The pharmaceutical composition of any one of claims 1 to 11, wherein the composition comprises Polysorbate-80 (PS80).

13. The pharmaceutical composition of claim 12, wherein the composition comprises PS80 at a concentration of 0.01% to 0.1%.

14. The pharmaceutical composition of claim 12, wherein the composition comprises PS80 at a concentration of 0.03% to 0.08%.

15. The pharmaceutical composition of claim 12, wherein the composition comprises PS80 at a concentration of 0.05%.

16. The pharmaceutical composition of any one of claims 1 to 15, wherein the composition comprises sodium citrate and citric acid.

17. The pharmaceutical composition of claim 16, wherein the composition comprises sodium citrate and citric acid at a concentration of 5 mM to 30 mM.

18. The pharmaceutical composition of claim 16, wherein the composition comprises sodium citrate and citric acid at a concentration of 15 mM to 25 mM.

19. The pharmaceutical composition of claim 16, wherein the composition comprises sodium citrate and citric acid at a concentration of 20 mM.

20. The pharmaceutical composition of any one of claims 1 to 19, wherein the composition has a pH of 5.3 to 5.6.

21. The pharmaceutical composition of any one of claims 1 to 19, wherein the composition has a pH of 5.5.

22. The pharmaceutical composition of any one of claims 1 to 21, wherein the composition comprises a thiol-containing antioxidant.

23. The pharmaceutical composition of claim 22, wherein the thiol-containing antioxidant is selected from the group consisting of GSH, GSSG, the combination of GSH and GSSG, cystine, cysteine, and the combination of cysteine and cystine.

24. The pharmaceutical composition of claim 22, wherein the thiol-containing antioxidant is GSH.

25. The pharmaceutical composition of claim 22, wherein the thiol-containing antioxidant is GSSG.

26. The pharmaceutical composition of claim 22, wherein the thiol-containing antioxidant is the combination of GSH and GSSG.

27. The pharmaceutical composition of any one of claims 22 to 26, wherein the thiol-containing antioxidant is at a concentration of 0.02 mM to 2 mM.

28. The pharmaceutical composition of any one of claims 22 to 26, wherein the thiol-containing antioxidant is at a concentration of 0.2 mM.

29. The pharmaceutical composition of any one of claims 22 to 26, wherein the thiol-containing antioxidant is at a concentration of 0.4 mM.

30. The pharmaceutical composition of any one of claims 22 to 26, wherein the thiol-containing antioxidant is at a concentration of 1 mM.

31. The pharmaceutical composition of claim 26, wherein the GSH is at a concentration of 0.4 mM and the GSSG is at a concentration of 0.2 mM.

32. The pharmaceutical composition of claim 1, comprising:
the anti-αvβ6 antibody or the αvβ6-binding fragment thereof at a concentration of 125 mg/ml to 175 mg/ml;
Arg.HCl at a concentration of 125 mM to 175 mM;
methionine at a concentration of 1 mM to 10 mM;
sodium citrate and citric acid at a concentration of 15 mM to 25 mM; and
PS80 at a concentration of 0.03% to 0.08%,
wherein the composition has a pH of 5.3 to 5.7.

33. The pharmaceutical composition of claim 1, comprising:
the anti-αvβ6 antibody or the αvβ6-binding fragment thereof at a concentration of 125 mg/ml to 175 mg/ml;
Arg.HCl at a concentration of 125 mM to 175 mM;
methionine at a concentration of 1 mM to 10 mM;
sodium citrate and citric acid at a concentration of 15 mM to 25 mM;
a thiol-containing antioxidant is a concentration of 0.02 mM to 2 mM; and
PS80 at a concentration of 0.03% to 0.08%,
wherein the composition has a pH of 5.3 to 5.7.

34. The pharmaceutical composition of claim 1, comprising:
the anti-αvβ6 antibody or the αvβ6-binding fragment thereof at a concentration of 150 mg/ml;
Arg.HCl at a concentration of 150 mM;
methionine at a concentration of 5 mM;
sodium citrate and citric acid at a concentration of 20 mM; and
PS80 at a concentration of 0.05%,
wherein the composition has a pH of 5.5.

35. The pharmaceutical composition of claim 1, comprising:
the anti-αvβ6 antibody or the αvβ6-binding fragment thereof at a concentration of 150 mg/ml;
Arg.HCl at a concentration of 150 mM;
methionine at a concentration of 5 mM;
sodium citrate and citric acid at a concentration of 20 mM;
a thiol-containing antioxidant selected from the group consisting of GSH at a concentration of 0.4 mM, cysteine at a concentration of 0.4 mM, GSSG at a concentration of 0.2 mM, cystine at a concentration of 0.2 mM, GSSH at a concentration of 0.2 mM and GSSG at a concentration of 0.4 mM, and cysteine at a concentration of 0.4 mM and cystine at a concentration of 0.2 mM; and
PS80 at a concentration of 0.05%,
wherein the composition has a pH of 5.5.

36. The pharmaceutical composition of any one of claims 1 to 35, wherein:
(i) the VH consists of a sequence at least 80% identical to SEQ ID NO:7 and the VL consists of a sequence at least 80% identical to SEQ ID NO:8;
(ii) the VH consists of a sequence at least 90% identical to SEQ ID NO:7 and the VL consists of a sequence at least 90% identical to SEQ ID NO:8; or
(iii) the VH consists of the amino acid sequence set forth in SEQ ID NO:7 and the VL consists of the amino acid sequence set forth in SEQ ID NO:8.

37. The pharmaceutical composition of any one of claims 1 to 36, wherein the anti-αvβ6 antibody comprises an immunoglobulin heavy chain and an immunoglobulin light chain, wherein:
(i) the heavy chain consists of a sequence at least 80% identical to SEQ ID NO:9 and the light chain consists of a sequence at least 80% identical to SEQ ID NO:10;
(ii) the heavy chain consists of a sequence at least 90% identical to SEQ ID NO:9 and the light chain consists of a sequence at least 90% identical to SEQ ID NO:10; or
(iii) the heavy chain consists of the amino acid sequence set forth in SEQ ID NO:9 and the light chain consists of the amino acid sequence set forth in SEQ ID NO:10.

38. A method of treating a condition selected from the group consisting of fibrosis, acute lung injury, and acute kidney injury in a human subject in need thereof, the method comprising administering to the human subject the pharmaceutical composition of any one of claims 1 to 37.

39. The method of claim 38, wherein the condition is fibrosis.

40. The method of claim 39, wherein the fibrosis is lung fibrosis.

41. The method of claim 40, wherein the lung fibrosis is idiopathic pulmonary fibrosis.

42. The method of any one of claims 38 to 41, wherein the pharmaceutical composition is administered subcutaneously to the human subject.

43. The method of any one of claims 38 to 42, wherein the anti-αvβ6 antibody or αvβ6-binding fragment thereof of the pharmaceutical composition is administered to the human subject at a dose of 40 mg once weekly.

44. The method of any one of claims 38 to 42, wherein the anti-αvβ6 antibody or αvβ6-binding fragment thereof of the pharmaceutical composition is administered to the human subject at a dose of 48 mg once weekly.

45. The method of any one of claims 38 to 42, wherein the anti-αvβ6 antibody or αvβ6-binding fragment thereof of the pharmaceutical composition is administered to the human subject at a dose of 56 mg once weekly.

46. The method of any one of claims 38 to 42, wherein the anti-αvβ6 antibody or αvβ6-binding fragment thereof of the pharmaceutical composition is administered to the human subject at a dose of 64 mg once weekly.

47. The method of any one of claims 38 to 42, wherein the anti-αvβ6 antibody or αvβ6-binding fragment thereof of the pharmaceutical composition is administered to the human subject at a dose of 0.5 mg/kg to 0.8 mg/kg once weekly.

48. The method of any one of claims 38 to 42, wherein the anti-αvβ6 antibody or αvβ6-binding fragment thereof of the pharmaceutical composition is administered to the human subject at a dose of 0.5 mg/kg once weekly.

49. The method of any one of claims 38 to 42, wherein the anti-αvβ6 antibody or αvβ6-binding fragment thereof of the pharmaceutical composition is administered to the human subject at a dose of 0.6 mg/kg once weekly.

50. The method of any one of claims 38 to 42, wherein the anti-αvβ6 antibody or αvβ6-binding fragment thereof of the pharmaceutical composition is administered to the human subject at a dose of 0.7 mg/kg once weekly.

51. The method of any one of claims 38 to 42, wherein the anti-αvβ6 antibody or αvβ6-binding fragment thereof of the pharmaceutical composition is administered to the human subject at a dose of 0.8 mg/kg once weekly.

52. A method of treating a condition selected from the group consisting of fibrosis, acute lung injury, and acute kidney injury in a human subject in need thereof, the method comprising administering subcutaneously to the human subject an anti-αvβ6 antibody or αvβ6-binding fragment thereof at a dose of 40 mg once every week, wherein the anti-αvβ6 antibody or αvβ6-binding fragment thereof comprises an immunoglobulin heavy chain variable domain (VH) and an immunoglobulin light chain variable domain (VL), the VH and VL, respectively, comprising:
(a) VH complementarity determining regions (CDRs), wherein
VH-CDR1 consists of the amino acid sequence set forth in SEQ ID NO:1;
VH-CDR2 consists of the amino acid sequence set forth in SEQ ID NO:2; and
VH-CDR3 consists of the amino acid sequence set forth in SEQ ID NO:3; and
(b) VL CDRs, wherein
VL-CDR1 consists of the amino acid sequence set forth in SEQ ID NO:4;
VL-CDR2 consists of the amino acid sequence set forth in SEQ ID NO:5; and
VL-CDR3 consists of the amino acid sequence set forth in SEQ ID NO:6.

53. A method of treating a condition selected from the group consisting of fibrosis, acute lung injury, and acute kidney injury in a human subject in need thereof, the method comprising administering subcutaneously to the human subject an anti-αvβ6 antibody or αvβ6-binding fragment thereof at a dose of 48 mg once every week, wherein the anti-αvβ6 antibody or αvβ6-binding fragment thereof comprises an immunoglobulin heavy chain variable domain (VH) and an immunoglobulin light chain variable domain (VL), the VH and VL, respectively, comprising:
(a) VH complementarity determining regions (CDRs), wherein
VH-CDR1 consists of the amino acid sequence set forth in SEQ ID NO:1;
VH-CDR2 consists of the amino acid sequence set forth in SEQ ID NO:2; and
VH-CDR3 consists of the amino acid sequence set forth in SEQ ID NO:3; and
(b) VL CDRs, wherein
VL-CDR1 consists of the amino acid sequence set forth in SEQ ID NO:4;
VL-CDR2 consists of the amino acid sequence set forth in SEQ ID NO:5; and
VL-CDR3 consists of the amino acid sequence set forth in SEQ ID NO:6.

54. A method of treating a condition selected from the group consisting of fibrosis, acute lung injury, and acute kidney injury in a human subject in need thereof, the method comprising administering subcutaneously to the human subject an anti-αvβ6 antibody or αvβ6-binding fragment thereof at a dose of 56 mg once every week, wherein the anti-αvβ6 antibody or αvβ6-binding fragment thereof comprises an immunoglobulin heavy chain variable domain (VH) and an immunoglobulin light chain variable domain (VL), the VH and VL, respectively, comprising:
(a) VH complementarity determining regions (CDRs), wherein
VH-CDR1 consists of the amino acid sequence set forth in SEQ ID NO:1;
VH-CDR2 consists of the amino acid sequence set forth in SEQ ID NO:2; and
VH-CDR3 consists of the amino acid sequence set forth in SEQ ID NO:3; and
(b) VL CDRs, wherein
VL-CDR1 consists of the amino acid sequence set forth in SEQ ID NO:4;
VL-CDR2 consists of the amino acid sequence set forth in SEQ ID NO:5; and
VL-CDR3 consists of the amino acid sequence set forth in SEQ ID NO:6.

55. A method of treating a condition selected from the group consisting of fibrosis, acute lung injury, and acute kidney injury in a human subject in need thereof, the method comprising administering subcutaneously to the human subject an anti-αvβ6 antibody or αvβ6-binding fragment thereof at a dose of 64 mg once every week, wherein the anti-αvβ6 antibody or αvβ6-binding fragment thereof comprises an immunoglobulin heavy chain variable domain (VH) and an immunoglobulin light chain variable domain (VL), the VH and VL, respectively, comprising:
(a) VH complementarity determining regions (CDRs), wherein
VH-CDR1 consists of the amino acid sequence set forth in SEQ ID NO:1;
VH-CDR2 consists of the amino acid sequence set forth in SEQ ID NO:2; and
VH-CDR3 consists of the amino acid sequence set forth in SEQ ID NO:3; and
(b) VL CDRs, wherein
VL-CDR1 consists of the amino acid sequence set forth in SEQ ID NO:4;
VL-CDR2 consists of the amino acid sequence set forth in SEQ ID NO:5; and
VL-CDR3 consists of the amino acid sequence set forth in SEQ ID NO:6.

56. The method of any one of claims 52 to 55, wherein the human subject is administered at least 4 doses of the anti-αvβ6 antibody or antigen-binding fragment thereof.

57. The method of any one of claims 52 to 55, wherein the human subject is administered at least 7 doses of the anti-αvβ6 antibody or antigen-binding fragment thereof.

58. The method of any one of claims 52 to 55, wherein the human subject is administered at least 10 doses of the anti-αvβ6 antibody or antigen-binding fragment thereof.

59. The method of any one of claims 52 to 58, wherein:
(i) the VH consists of a sequence at least 80% identical to SEQ ID NO:7 and the VL consists of a sequence at least 80% identical to SEQ ID NO:8;
(ii) the VH consists of a sequence at least 90% identical to SEQ ID NO:7 and the VL consists of a sequence at least 90% identical to SEQ ID NO:8; or
(iii) the VH consists of the amino acid sequence set forth in SEQ ID NO:7 and the VL consists of the amino acid sequence set forth in SEQ ID NO:8.

60. The method of any one of claims 52 to 59, wherein the anti-αvβ6 antibody comprises an immunoglobulin heavy chain and an immunoglobulin light chain, wherein:
(i) the heavy chain consists of a sequence at least 80% identical to SEQ ID NO:9 and the light chain consists of a sequence at least 80% identical to SEQ ID NO:10;
(ii) the heavy chain consists of a sequence at least 90% identical to SEQ ID NO:9 and the light chain consists of a sequence at least 90% identical to SEQ ID NO:10; or
(iii) the heavy chain consists of the amino acid sequence set forth in SEQ ID NO:9 and the light chain consists of the amino acid sequence set forth in SEQ ID NO:10.

61. The method of any one of claims 52 to 60, wherein the condition is fibrosis.

62. The method of claim 61, wherein the fibrosis is lung fibrosis.

63. The method of claim 62, wherein the lung fibrosis is idiopathic pulmonary fibrosis.

64. A syringe or pump comprising a sterile preparation of the pharmaceutical composition of any one of claims 1 to 37 adapted for subcutaneous administration of the anti-αvβ6 antibody or αvβ6-binding fragment thereof at a fixed dose of 40 mg, 48 mg, 56 mg, or 64 mg.

65. A syringe or pump comprising 0.5 to 5.0 mL of a sterile preparation of the pharmaceutical composition of any one of claims 1 to 37.

66. A syringe or pump comprising a sterile preparation of an anti-αvβ6 antibody or αvβ6-binding fragment thereof, wherein the syringe or pump is adapted for subcutaneous administration of the anti-αvβ6 antibody or αvβ6-binding fragment thereof at a fixed dose of 40 mg, 48 mg, 56 mg, or 64 mg, and wherein the anti-αvβ6 antibody or αvβ6-binding fragment thereof comprises an immunoglobulin heavy chain variable domain (VH) and an immunoglobulin light chain variable domain (VL), the VH and VL, respectively, comprising:
(a) VH complementarity determining regions (CDRs), wherein
VH-CDR1 consists of the amino acid sequence set forth in SEQ ID NO:1;
VH-CDR2 consists of the amino acid sequence set forth in SEQ ID NO:2; and
VH-CDR3 consists of the amino acid sequence set forth in SEQ ID NO:3; and
(b) VL CDRs, wherein
VL-CDR1 consists of the amino acid sequence set forth in SEQ ID NO:4;
VL-CDR2 consists of the amino acid sequence set forth in SEQ ID NO:5; and
VL-CDR3 consists of the amino acid sequence set forth in SEQ ID NO:6.

67. The syringe or pump of claim 66, wherein:
(i) the VH consists of a sequence at least 80% identical to SEQ ID NO:7 and the VL consists of a sequence at least 80% identical to SEQ ID NO:8;
(ii) the VH consists of a sequence at least 90% identical to SEQ ID NO:7 and the VL consists of a sequence at least 90% identical to SEQ ID NO:8; or
(iii) the VH consists of the amino acid sequence set forth in SEQ ID NO:7 and the VL consists of the amino acid sequence set forth in SEQ ID NO:8.

68. The syringe or pump of claim 66 or claim 67, wherein the anti-αvβ6 antibody comprises an immunoglobulin heavy chain and an immunoglobulin light chain, wherein:
(i) the heavy chain consists of a sequence at least 80% identical to SEQ ID NO:9 and the light chain consists of a sequence at least 80% identical to SEQ ID NO:10;
(ii) the heavy chain consists of a sequence at least 90% identical to SEQ ID NO:9 and the light chain consists of a sequence at least 90% identical to SEQ ID NO:10; or
(iii) the heavy chain consists of the amino acid sequence set forth in SEQ ID NO:9 and the light chain consists of the amino acid sequence set forth in SEQ ID NO:10.

69. The method of any one of claims 38 to 63, wherein the method further comprises administering to the human subject a therapeutically effective amount of prifenidone or nintedanib.

70. The method of claim 69, wherein the human subject is administered prifenidone as follows:

| Treatment days | Dosage |
|---|---|
| Days 1 through 7 | 267 mg three times daily (801 mg/day) |
| Days 8 through 14 | 534 mg three times daily (1602 mg/day) |
| Days 15 onward | 801 mg three times daily (2403 mg/day). |

71. The method of claim 69, wherein the human subject is administered nintedanib at a fixed dose of 150 mg twice daily.


SEQUENCE LISTING (13 sequences)

SEQ ID NO: 1 - protein, 5 aa, Artificial Sequence
  source: /note=“Description of Artificial Sequence Synthetic peptide”
RYVMS

SEQ ID NO: 2 - protein, 16 aa, Artificial Sequence
  source: /note=“Description of Artificial Sequence Synthetic peptide”
SISSGGRMYYPDTVKG

SEQ ID NO: 3 - protein, 12 aa, Artificial Sequence
  source: /note=“Description of Artificial Sequence Synthetic peptide”
GSIYDGYYVFPY

SEQ ID NO: 4 - protein, 12 aa, Artificial Sequence
  source: /note=“Description of Artificial Sequence Synthetic peptide”
SASSSVSSSYLY

SEQ ID NO: 5 - protein, 7 aa, Artificial Sequence
  source: /note=“Description of Artificial Sequence Synthetic peptide”
STSNLAS

SEQ ID NO: 6 - protein, 9 aa, Artificial Sequence
  source: /note=“Description of Artificial Sequence Synthetic peptide”
HQWSTYPPT

SEQ ID NO: 7 - protein, 120 aa, Artificial Sequence
  source: /note=“Description of Artificial Sequence Synthetic polypeptide”
EVQLVESGGGLVQPGGSLRLSCAASGFTFSRYVMSWVRQAPGKGLEWVASISSGGRMYYPDTVKGRFTISRDNAKNSLYLQMNSLRAEDTAVYYCARGSIYDGYYVFPYWGQGTLVTVSS

SEQ ID NO: 8 - protein, 108 aa, Artificial Sequence
  source: /note=“Description of Artificial Sequence Synthetic polypeptide”
EIVLTQSPATLSLSPGERATLSCSASSSVSSSYLYWYQQKPGQAPRLLIYSTSNLASGIPARFSGSGSGTDFTLTISSLEPEDFAVYYCHQWSTYPPTFGGGTKVEIK

SEQ ID NO: 9 - protein, 449 aa, Artificial Sequence
  source: /note=“Description of Artificial Sequence Synthetic polypeptide”
EVQLVESGGGLVQPGGSLRLSCAASGFTFSRYVMSWVRQAPGKGLEWVASISSGGRMYYPDTVKGRFTISRDNAKNSLYLQMNSLRAEDTAVYYCARGSIYDGYYVFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 215 aa, Artificial Sequence
  source: /note=“Description of Artificial Sequence Synthetic polypeptide”
EIVLTQSPATLSLSPGERATLSCSASSSVSSSYLYWYQQKPGQAPRLLIYSTSNLASGIPARFSGSGSGTDFTLTISSLEPEDFAVYYCHQWSTYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 10 aa, Artificial Sequence
  source: /note=“Description of Artificial Sequence Synthetic peptide”
GFTFSRYVMS

SEQ ID NO: 12 - protein, 1048 aa, Homo sapiens
MAFPPRRRLRLGPRGLPLLLSGLLLPLCRAFNLDVDSPAEYSGPEGSYFGFAVDFFVPSASSRMFLLVGAPKANTTQPGIVEGGQVLKCDWSSTRRCQPIEFDATGNRDYAKDDPLEFKSHQWFGASVRSKQDKILACAPLYHWRTEMKQEREPVGTCFLQDGTKTVEYAPCRSQDIDADGQGFCQGGFSIDFTKADRVLLGGPGSFYWQGQLISDQVAEIVSKYDPNVYSIKYNNQLATRTAQAIFDDSYLGYSVAVGDFNGDGIDDFVSGVPRAARTLGMVYIYDGKNMSSLYNFTGEQMAAYFGFSVAATDINGDDYADVFIGAPLFMDRGSDGKLQEVGQVSVSLQRASGDFQTTKLNGFEVFARFGSAIAPLGDLDQDGFNDIAIAAPYGGEDKKGIVYIFNGRSTGLNAVPSQILEGQWAARSMPPSFGYSMKGATDIDKNGYPDLIVGAFGVDRAILYRARPVITVNAGLEVYPSILNQDNKTCSLPGTALKVSCFNVRFCLKADGKGVLPRKLNFQVELLLDKLKQKGAIRRALFLYSRSPSHSKNMTISRGGLMQCEELIAYLRDESEFRDKLTPITIFMEYRLDYRTAADTTGLQPILNQFTPANISRQAHILLDCGEDNVCKPKLEVSVDSDQKKIYIGDDNPLTLIVKAQNQGEGAYEAELIVSIPLQADFIGVVRNNEALARLSCAFKTENQTRQVVCDLGNPMKAGTQLLAGLRFSVHQQSEMDTSVKFDLQIQSSNLFDKVSPVVSHKVDLAVLAAVEIRGVSSPDHVFLPIPNWEHKENPETEEDVGPVVQHIYELRNNGPSSFSKAMLHLQWPYKYNNNTLLYILHYDIDGPMNCTSDMEINPLRIKISSLQTTEKNDTVAGQGERDHLITKRDLALSEGDIHTLGCGVAQCLKIVCQVGRLDRGKSAILYVKSLLWTETFMNKENQNHSYSLKSSASFNVIEFPYKNLPIEDITNSTLVTTNVTWGIQPAPMPVPVWVIILAVLAGLLLLAVLVFVMYRMGFFKRVRPPQEEQEREQLQPHENGEGNSET

SEQ ID NO: 13 - protein, 788 aa, Homo sapiens
MGIELLCLFFLFLGRNDHVQGGCALGGAETCEDCLLIGPQCAWCAQENFTHPSGVGERCDTPANLLAKGCQLNFIENPVSQVEILKNKPLSVGRQKNSSDIVQIAPQSLILKLRPGGAQTLQVHVRQTEDYPVDLYYLMDLSASMDDDLNTIKELGSRLSKEMSKLTSNFRLGFGSFVEKPVSPFVKTTPEEIANPCSSIPYFCLPTFGFKHILPLTNDAERFNEIVKNQKISANIDTPEGGFDAIMQAAVCKEKIGWRNDSLHLLVFVSDADSHFGMDSKLAGIVIPNDGLCHLDSKNEYSMSTVLEYPTIGQLIDKLVQNNVLLIFAVTQEQVHLYENYAKLIPGATVGLLQKDSGNILQLIISAYEELRSEVELEVLGDTEGLNLSFTAICNNGTLFQHQKKCSHMKVGDTASFSVTVNIPHCERRSRHIIIKPVGLGDALELLVSPECNCDCQKEVEVNSSKCHHGNGSFQCGVCACHPGHMGPRCECGEDMLSTDSCKEAPDHPSCSGRGDCYCGQCICHLSPYGNIYGPYCQCDNFSCVRHKGLLCGGNGDCDCGECVCRSGWTGEYCNCTTSTDSCVSEDGVLCSGRGDCVCGKCVCTNPGASGPTCERCPTCGDPCNSKRSCIECHLSAAGQAREECVDKCKLAGATISEEEDFSKDGSVSCSLQGENECLITFLITTDNEGKTIIHSINEKDCPKPPNIPMIMLGVSLAILLIGVVLLCIWKLLVSFHDRKEVAKFEAERSKAKWQTGTNPLYRGSTSTFKNVTYKHREKQKVDLSTDC